(19) United States
(12) Patent Application Publication
(10) Pub. No.: US 2023/0302090 A1
(43) Pub. Date: Sep. 28, 2023

(54) COMBINATION THERAPY FOR TREATMENT OF CANCER

(71) Applicant: ONCOSEC MEDICAL INCORPORATED, Pennington, NJ (US)

(72) Inventor: Christopher G. TWITTY, San Diego, CA (US)

(21) Appl. No.: 18/018,532

(22) PCT Filed: Jul. 29, 2021
(86) PCT No.: PCT/US2021/043665

§ 371 Date: Jan. 27, 2023

Related U.S. Application Data
(60) Provisional application No. 63/058,026, filed on Jul. 29, 2020.

(51) Int. Cl. A61K 38/20; A61K 39/395; A61K 31/337; A61P 35/00; A61P 35/04; A61N 1/32

(52) U.S. Cl. CPC A61K 38/208; A61K 39/3955; A61K 31/337; A61P 35/00; A61P 35/04; A61N 1/327; A61K 2039/505

(57) ABSTRACT

Described are methods and treatment schedules for treating cancer with intratumoral electroporation of an immunostimulatory cytokine combined with administration of an immune checkpoint inhibitor and a chemotherapeutic agent.

### CROSS-REFERENCE TO RELATED APPLICATIONS

This application claims the benefit of U.S. Provisional Application No. 63/058,026, filed Jul. 29, 2020, which is incorporated herein by reference.

### BACKGROUND

Breast cancer is the most common cancer diagnosed among US women and is the second leading cause of cancer-related deaths. Triple negative-breast cancer (TNBC) represents 1 of the 4 main molecular subtypes of invasive breast cancer accounting for 10-20% of total cases. TNBC is biologically heterogeneous but can be mainly identified by a negative phenotype for the estrogen (ER) and progesterone (PR) receptors and a lack of gene amplification/protein overexpression for the human epidermal growth factor receptor 2 (HER2). These biologic characteristics confer a higher aggressiveness and relapse risk than that observed in all other breast cancer subtypes.

Due to the loss of the tumor cell receptors, patients with TNBC do not benefit from hormonal therapy or treatments targeting the oncogenic HER2 pathway. Chemotherapy is the current standard-of-care treatment in the adjuvant, neoadjuvant, and metastatic settings. The standard of care for patients with recurrent and/or metastatic disease is cytotoxic chemotherapy Median survival is approximately 13 months from the time of recurrence or diagnosis of distant metastases. Thus, improved methods of treating TNBC are needed.

### SUMMARY

Described herein are methods of treating a cancer comprising administering to a subject a therapeutically effective amount of an immunostimulatory cytokine in combination with a checkpoint inhibitor and a chemotherapeutic agent.

In some embodiments, methods of treating a subject having cancer are described, the methods comprising:
a) administering to at least one tumor in the subject an effective dose of at least one expression vector coding for at least one immunostimulatory cytokine by intratumoral electroporation;
b) administering an effective dose of an immune checkpoint inhibitor to the subject; and
c) administering an effective dose of a chemotherapeutic agent to the subject.
   The cancer can be stage 0, stage I, stage II, stage III, or stage IV. The cancer can be operable or inoperable. The cancer can be locally advanced cancer (stage II or III), recurrent, or metastatic (stage IV). The cancer can be, but is not limited to, breast cancer, triple negative breast cancer, melanoma, head and neck cancer, squamous cell carcinoma, basal cell carcinoma, and Merkel cell carcinoma. In some embodiments, the cancer is operable. In some embodiments, the cancer in inoperable. In some embodiments, the cancer in inoperable locally advanced or metastatic cancer.

In some embodiments, a methods of treating a subject having TNBC are described, the methods comprising:
a) administering to at least one tumor in the subject an effective dose of at least one expression vector coding for at least one immunostimulatory cytokine by intratumoral electroporation;
b) administering an effective dose of an immune checkpoint inhibitor to the subject; and
c) administering an effective dose of a chemotherapeutic agent to the subject.
   The TNBC can be stage 0, stage I, stage II, stage III, or stage IV. The TNBC can be operable or inoperable. The cancer can be locally advanced cancer (stage II or III), recurrent, or metastatic (stage IV). In some embodiments, the TNBC is operable. In some embodiments, the TNBC in inoperable. In some embodiments, the TNBC in inoperable locally advanced or metastatic TNBC.

In some embodiments, first line treatment methods for treating a subject having cancer are described, the methods comprising:
a) administering to at least one tumor in the subject an effective dose of at least one expression vector coding for at least one immunostimulatory cytokine by intratumoral electroporation;
b) administering an effective dose of an immune checkpoint inhibitor to the subject; and
c) administering an effective dose of a chemotherapeutic agent to the subject.
   The cancer can be stage 0, stage I, stage II, stage III, or stage IV. The cancer can be operable or inoperable. The cancer can be locally advanced cancer (stage II or III), recurrent, or metastatic (stage IV). The cancer can be, but is not limited to, breast cancer, triple negative breast cancer, melanoma, head and neck cancer, squamous cell carcinoma, basal cell carcinoma, and Merkel cell carcinoma. In some embodiments, the cancer is operable. In some embodiments, the cancer in inoperable. In some embodiments, the cancer in inoperable locally advanced or metastatic cancer.

In some embodiments, first line treatment methods for treating a subject having TNBC are described, the methods comprising:
a) administering to at least one tumor in the subject an effective dose of at least one expression vector coding for at least one immunostimulatory cytokine by intratumoral electroporation;
b) administering an effective dose of an immune checkpoint inhibitor to the subject; and
c) administering an effective dose of a chemotherapeutic agent to the subject.
   The TNBC can be stage 0, stage I, stage II, stage III, or stage IV. The TNBC can be operable or inoperable. The cancer can be locally advanced cancer (stage II or III), recurrent, or metastatic (stage IV). In some embodiments, the TNBC is operable. In some embodiments, the TNBC in inoperable. In some embodiments, the TNBC in inoperable locally advanced or metastatic TNBC.

In some embodiments, second line treatment methods for treating a subject having cancer are described, the methods comprising:
a) administering to at least one tumor in the subject an effective dose of at least one expression vector coding for at least one immunostimulatory cytokine by intratumoral electroporation;
b) administering an effective dose of an immune checkpoint inhibitor to the subject; and
c) administering an effective dose of a chemotherapeutic agent to the subject.
   The cancer can be stage 0, stage I, stage II, stage III, or stage IV. The cancer can be operable or inoperable. The cancer can be locally advanced cancer (stage II or III), recurrent, or metastatic (stage IV). The cancer can be, but is not limited to, breast cancer, triple negative breast cancer, melanoma, head and neck cancer, squamous cell carcinoma, basal cell carcinoma, and Merkel cell carcinoma. In some embodiments, the cancer is operable. In some embodiments, the cancer in inoperable. In some embodiments, the cancer in inoperable locally advanced or metastatic cancer

In some embodiments, second line treatment methods for treating a subject having TNBC are described, the methods comprising:
a) administering to at least one tumor in the subject an effective dose of at least one expression vector coding for at least one immunostimulatory cytokine by intratumoral electroporation;
b) administering an effective dose of an immune checkpoint inhibitor to the subject; and
c) administering an effective dose of a chemotherapeutic agent to the subject.
   The TNBC can be stage 0, stage I, stage II, stage III, or stage IV. The TNBC can be operable or inoperable. The cancer can be locally advanced cancer (stage II or III), recurrent, or metastatic (stage IV). In some embodiments, the TNBC is operable. In some embodiments, the TNBC in inoperable. In some embodiments, the TNBC in inoperable locally advanced or metastatic TNBC.

The immunostimulatory cytokine may be selected from the group consisting of: IL-1, IL-2, IL-7, IL-10, IL-12, IL-15, IL-15/Receptor α, IL-21, IL-23, IL-27, IL-35, IFN-α, IFN-β, IFN-γ, TNF-α, TGF-β, and C-X-C Motif Chemokine ligand 9 (CXCL9). In some embodiments, the immunostimulatory cytokine comprises IL-12.

Intratumoral electroporation comprises injection of the expression vector into the tumor and administering at least one electroporative pulse can to the tumor. The electroporative pulse can comprise administrating of at least one voltage pulse over a duration of about 100 microseconds to about 1 millisecond and a field strength of about 300V/cm to about 1500V/cm.

In some embodiments, the immune checkpoint inhibitor is administered systemically. Systemic injection can comprise intravenous infusion. In some embodiments, the immune checkpoint inhibitor is encoded on an expression vector and delivered to the cancerous tumor by electroporation therapy. In some embodiments, the immune checkpoint inhibitor is encoded on an expression vector that also encodes the immunostimulatory cytokine and delivered to the cancerous tumor by electroporation therapy. The immune checkpoint inhibitor is an antagonist of at least one immune checkpoint protein of Table 1. The immune checkpoint inhibitor can be, but is not limited to, an anti-PD-1 antibody, an anti-PD-L1 antibody, an anti-PD-L2 antibody, or an anti-CTLA-4 1 antibody. The immune checkpoint inhibitor can be, but is not limited to, nivolumab, pembrolizumab, pidilizumab, durvalumab, atezolizumab, avelumab, cemiplimab, sintilimab, toripalimab, or camrelizumab. In some embodiments, the immune checkpoint inhibitor comprises a PD-1, PD-L1 or PD-L2 antagonist. In some embodiments, the immune checkpoint inhibitor is administered after electroporation of the immunostimulatory cytokine. In some embodiments, immune checkpoint inhibitor is administered before electroporation of the immunostimulatory cytokine. In some embodiments, immune checkpoint inhibitor is administered essentially concurrently with electroporation of the immunostimulatory cytokine. One days in which the immune checkpoint inhibitor and immunostimulatory cytokine are administered on the same day, immune checkpoint inhibitor may be administered prior to, concurrently with, or after administration of IT-EP immunostimulatory cytokine. In some embodiments, IT-EP immunostimulatory cytokine is administered prior to administration of immune checkpoint inhibitor. One days in which chemotherapeutic and IT-EP immunostimulatory cytokine are administered on the same day, chemotherapeutic may be administered prior to, concurrently with, or after administration of IT-EP immunostimulatory cytokine. One days in which immune checkpoint inhibitor and chemotherapeutic are administered on the same day, immune checkpoint inhibitor may be administered prior to, concurrently with, or after administration of chemotherapeutic.

The chemotherapeutic agent can be any chemotherapeutic approved or authorized for the treatment of the cancer. In some embodiments, such as for treatment of triple negative breast cancer, the chemotherapeutic is approved or authorized for the treatment of breast cancer or triple negative breast cancer. The chemotherapeutic can be, but is not limited to, an anthracycline (e.g., daunorubicin, doxorubicin, pegylated liposomal doxorubicin, epirubicin), a cyclophosphamide, an alkylating agent (e.g., thiotepa), a taxane (e.g., docetaxel, paclitaxel, nab-paclitaxel), a nucleotide analog or antimetabolite (e.g., fluorouracil (5-FU), gemcitabine, methotrexate, capecitabine), a microtubule inhibitor (e.g., eribulin), platinum agent (e.g., cisplatin, carboplatin), a PI3K inhibitor (e.g., alpelisib), a poly ADP-ribose polymerase (PARP) inhibitor (e.g., olaparib, talazoparib), cytoxan, ixabepilone, mutanycin, vinorelbine, or combinations thereof. In some embodiments, the cancer is breast cancer or TNBC and the chemotherapeutic agent comprises a taxane such as paclitaxel or nab-paclitaxel.

Each of the immunostimulatory cytokine, immune checkpoint inhibitor, and chemotherapeutic are administered to the subject in cycles. For administering the effective dose of the at least one expression vector coding for the at least one immunostimulatory cytokine by intratumoral electroporation, the expression vector is administered by IT-EP on day 1 (±2 days); days 1 (±2 days) and 5 (±2 days); days 1 (±2 days) and 8 (±2 days); or days 1 (±2 days), 5 (±2 days), and 8 (±2 days) of a 3-6 week cycle (i.e., on the indicated days every 3-6 six weeks for the duration for the treatment). In some embodiments, the immunostimulatory cytokine is administered according to a six week cycle. In some embodiments, the immunostimulatory cytokine administered on days 1 (±2 days), 5 (±2 days), and 8 (±2 days) of a 6 week cycle. In some embodiments, the immunostimulatory cytokine administered on days 1 (±2 days), 8 (±2 days), and 15 (±2 days) of a 6 week cycle. In some embodiments, the immunostimulatory cytokine administered on days 1 (±2 days) and 8 (±2 days) of a 4 week cycle. For administering the effective dose of the immune checkpoint inhibitor, the immune checkpoint inhibitor is administered on day 1 (±2 days) of a 3-6 week cycle (i.e., on the indicated day every 3-6 six weeks for the duration for the treatment). In some embodiments, the immune checkpoint inhibitor is administered on day 1 (±2 days) of a 3 week cycle. In some embodiments, the immune checkpoint inhibitor is administered on day 1 (±2 days) of a 4 week cycle. The chemotherapeutic can be administered to the subject according to generally accepted practices (i.e., according to the product label or generally accepted standard of care) for the chemotherapeutic. In some embodiments, the chemotherapeutic is administered to the subject on day 1 (±2 days); days 1 (±2 days) and 8 (±2 days); days 1 (±2 days) and 15 (±2 days); or days 1 (±2 days), 8 (±2 days), and 15 (±2 days) of a 1-6 week cycle (i.e., on the indicated days every 1-6 weeks for the duration for the treatment). In some embodiments, the immune checkpoint inhibitor is administered according to a four week cycle. In some embodiments, the immune checkpoint inhibitor is administered on days 1, 8 and 15 of a 4 week cycle. The treatment cycles for each of the immunostimulatory cytokine, immune checkpoint inhibitor, and chemotherapeutic are administered concurrently. In some embodiments, treatment cycles for each of the immunostimulatory cytokine, immune checkpoint inhibitor, and chemotherapeutic are initiated on the same day, such that, for example day of a three week immunostimulatory cytokine treatment cycle, day one of a immune checkpoint inhibitor treatment cycle, and day 1 of a chemotherapeutic treatment cycle begin on the same day. In some embodiments, the immunostimulatory cytokine comprises IIL-12, the checkpoint inhibitor comprise a PD-1 or PD-L1 antagonist, and the chemotherapeutic comprises nab-paclitaxel.

In some embodiments, methods of cancer in a subject are described comprising:
(a) injecting a tumor in the subject with an effective dose of at least one expression vector coding for at least one immunostimulatory cytokine and administering electroporation therapy to the tumor and days 1, 5, and 8 of a 6 week cycle;
(b) administering an effective dose of an immune checkpoint inhibitor to the subject on day 1 of a 3 week cycle; and
(c) administering an effective dose of a chemotherapeutic agent to the subject on days 1, 8, and 15 of a 4 week cycle.
   The subject can be treated with multiple cycles of each agent. The cycles for each agent (immunostimulatory cytokine, immune checkpoint inhibitor, and chemotherapeutic) can run consecutively, i.e., every 6 weeks, every 3 weeks and every 4 weeks, respectively. In some embodiments, day 1 for the initial cycle for each agent begins on the same day and the cycles run concurrently. In some embodiments, the immunostimulatory cytokine, immune checkpoint inhibitor, and chemotherapeutic are administered to the subject according to the treatment schedule diagramed in FIG. 1. In some embodiments, the immunostimulatory cytokine comprises IL-12, the checkpoint inhibitor comprise a PD-1 or PD-L1 antagonist (i.e., anti-PD-1 antibody or anti-PD-L1 antibody), and the chemotherapeutic comprises nab-paclitaxel.

Also described are combination therapies comprising an immunostimulatory cytokine, a checkpoint inhibitor and the chemotherapeutic formulated for administration according to any of the described methods.

The methods and combinations can be used to treat a subject having inoperable cancer. The inoperable cancer can be locally advanced or metastatic. The inoperable cancer, locally advanced or metastatic cancer can be, but is not limited to TNBC. In some embodiments, the subjects has received one or more prior cancer therapies. In some embodiments, the subject has not received one or more prior cancer therapies. In some embodiments, method can be used to treat a subject that has received prior neoadjuvant or adjuvant treatment in the non-metastatic or potentially operable disease setting.

The methods and combinations can be used to treat a subject having advanced, metastatic, treatment refractory cancer or tumor. A treatment refractory cancer or tumor can be, but is not limited to, an immune checkpoint inhibitor refractory cancer tumor, a hormone refractory cancer or tumor, a radiation refractory cancer tumor, or a chemotherapy refractory cancer or tumor. In some embodiments, the subject has failed to respond to at least one course of immune checkpoint inhibitor therapy. In some embodiments, the subject is progressing on or has progressed on one or more anti-cancer therapies, such as, but not limited to, checkpoint inhibitor therapy.

The methods and combinations can be used to treat subjects having tumors predicted to be refractory to or not respond to one or more anti-cancer therapies. In some embodiments, the subject has low tumor infiltrating lymphocytes, low partially cytotoxic lymphocytes, or exhausted T cells. In some embodiments, the subject has advanced on one or more prior cancer therapies.

### BRIEF DESCRIPTION OF THE DRAWINGS

FIG. 1. Exemplary treatment schedule.

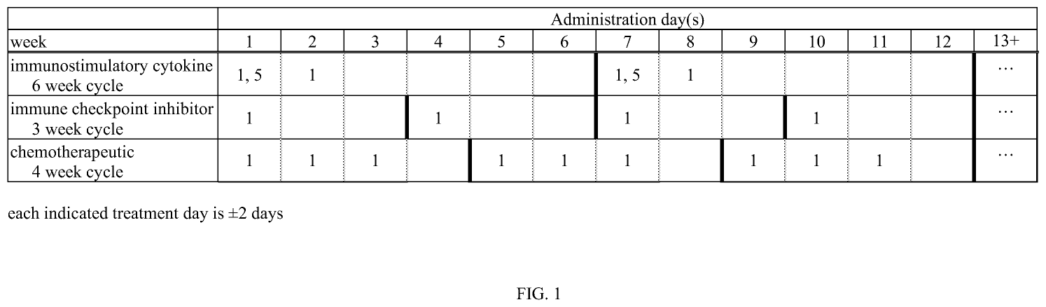

### DETAILED DESCRIPTION

As used herein, including the appended claims, the singular forms of words such as “a,” “an,” and “the,” include their corresponding plural references unless the context clearly dictates otherwise. The conjunction “or” is to be interpreted in the inclusive sense, i.e., as equivalent to “and/or,” unless the inclusive sense would be unreasonable in the context. Use of “comprise,” “comprises,” “comprising,” “contain,” “contains,” “containing,” “include,” “includes,” and “including” are not intended to be limiting. It is to be understood that both the foregoing general description and detailed description are exemplary and explanatory only and are not restrictive of the teachings.

In general, the term “about” indicates insubstantial variation in a quantity of a component of a composition not having any significant effect on the activity or stability of the composition. The term “about” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 0 to 20%, 0 to 10%, 0 to 5%, or up to 1% of a given value. Where particular values are described in the application and claims, unless otherwise stated the term “about” meaning within an acceptable error range for the particular value should be assumed. All ranges are to be interpreted as encompassing the endpoints in the absence of express exclusions such as “not including the endpoints”; thus, for example, “within 10-15” includes the values 10 and 15. One skilled in the art will understand that the recited ranges include the end values, as whole numbers in between the end values, and where practical, rational numbers within the range (e.g., the range 5-10 includes 5, 6, 7, 8, 9, and 10, and where practical, values such as 6.8, 9.35, etc.).

All references cited herein are incorporated by reference to the same extent as if each individual publication, patent application, or patent, was specifically and individually indicated to be incorporated by reference.

### A. Definitions

An “immune checkpoint protein” is any one of a group of immune cell surface receptor/ligands which induce T cell dysfunction or apoptosis. These immune inhibitory targets attenuate excessive immune reactions and ensure self-tolerance. Tumor cells harness the suppressive effects of these checkpoint proteins. Immune checkpoints are a normal part of the immune system. Their role is to prevent uncontrolled immune reactions. Immune checkpoints engage when receptors on the surface of T cells recognize and bind to checkpoint proteins expressed by other cells in a process called T cell exhaustion. Some tumors evade immune response by expressing these checkpoint proteins. Examples of checkpoint proteins include, but are not limited to, Cytotoxic T Lymphocyte Antigen-4 (CTLA-4), Programmed Death 1 (PD-1), Programmed Death Ligand 1 (PD-L1), PD-L2, Lymphocyte Activation Gene-3 (LAG-3), T cell Immunoglobulin Mucin-3 (TIM3), Killer Cell Immunoglobulin-like Receptor (MR), B- and T-Lymphocyte Attenuator (BTLA), Adenosine A2a Receptor (A2aR), and Herpes Virus Entry Mediator (HVEM). Exemplary immune checkpoint proteins are listed in Table 1. Blocking interaction between checkpoint proteins and their T cell receptors using checkpoint inhibitors is used to overcome T cells exhaustion thereby increasing immune response against a tumor.

TABLE 1
Immune Checkpoint Proteins

| | | GenBank | GenBank | GenBank | GenBank |
|---|---|---|---|---|---|
| | | Accession | Accession | Accession | Accession |
| | Unabbreviated | Number - Mouse | Number - Mouse | Number - Human | Number - Human |
| Target | Name | Nucleic Acid | Amino Acid | Nucleic Acid | Amino Acid |
| CTLA-4 | Cytotoxic T | U90271 | AAD00697 | L15006 | AAL07473 |
| | Lymphocyte | | | | |
| | Antigen-4 | | | | |
| PD-1 | Programmed | NM_008798.2 | MP_032824 | NM_005018 | NP_005009.2 |
| | Death 1 | | | | |
| PD-L1 | Programmed | GQ904197 | ADK70950 | AY254342 | AAP13470 |
| | Death Ligand 1 | | | | |
| LAG-3 | Lymphocyte | AY230414 | AAP57397 | X51985 | CAA36243 |
| | Activation | | | | |
| | Gene-3 | | | | |
| TIM3 | T cell | AF450241 | AAL35776 | JX049979 | AF066593 |
| | Immunoglobulin | | | | |
| | Mucin-3 | | | | |
| KIR | Killer Cell | AY130461 | AY130461.1 | AY601812 | AAT11793 |
| | Immunoglobulin- | | | | |
| | like Receptor | | | | |
| BTLA | B- and T- | AY293285 | AAP44002 | AY293286 | AAP44003 |
| | Lymphocyte | | | | |
| | Attenuator | | | | |
| A2aR | Adenosine A2a | NM_009630 | NP_033760 | NP_001265428 | NM_001278499 |
| | Receptor | | | | |
| HVEM | Herpes Virus | AF515707 | AAQ08183 | AY358879 | AAQ89238 |
| | Entry Mediator | | | | |

An “immune checkpoint inhibitor” (checkpoint inhibitor) is a molecule that inhibits or prevents immune suppression by blocking the effects of an immune checkpoint protein. Checkpoint inhibitors include, but are not limited to, antibodies and antibody fragments, nanobodies, diabodies, soluble binding partners of checkpoint molecules, small molecule therapeutics, and peptide antagonists. Examples of checkpoint inhibitors include anti-checkpoint protein antibodies. An immune checkpoint inhibitor can be, but is not limited to, a PD-1 and/or PD-L1 antagonist. A PD-1 and/or PD-L1 antagonist can be, but is not limited to, an anti-PD-1 or anti-PD-L1 antibody. Anti-PD-1/PD-L1 antibodies include, but are not limited to, nivolumab, pembrolizumab, pidilizumab, and atezolizumab.

An “immunostimulatory cytokine” includes cytokines that mediate or enhance the immune response to a foreign antigen, including viral, bacterial, or tumor antigens. Innate immunostimulatory cytokines can include, e.g., IL-1, IL-2, IL-7, IL-10, IL-12, IL-15, IL-15/Receptor α, IL-21, IL-23, IL-27, IL-35, IFN-α, IFN-β, IFN-γ, TNF-α, TGF-β, and CXCL9. Adaptive immunostimulatory cytokines include, e.g., IL-2, IL-4, IL-5, TGF-β, IL-10 and IFN-γ. Examples of immunostimulatory cytokines are provided in Table 2.

TABLE 2
Immunostimulatory Cytokines

| | GenBank | GenBank | GenBank | GenBank |
|---|---|---|---|---|
| | Accession | Accession | Accession | Accession |
| | Number - Mouse | Number - Mouse | Number - Human | Number - Human |
| Cytokine | Nucleic Acid | Amino Acid | Nucleic Acid | Amino Acid |
| TNF-α | M20155 | CAA68530 | X02910 | ADV31546 |
| IL-1 | RN U48592 | CAA28637 | X03833 | CAA27448 |
| IL-10 | M84340.1 | AAA39275 | U16720.1 | AAA80104 |
| IL-12 | | | | AAD16432 |
| p35 | NM_001159424.2 | NP_001152896.1 | NM_000882.3 | NP_000873.2 |
| p40 | NM_001303244.1 | NP_001290173.1 | NM_002187.2 | NP_002178.2 |
| IL-15 | NM_001254747.1 | NP_001241676 | NM_000585.4 | NP_000576 |
| IL-15Ra | NM_008358.2 | NP_032384 | NM_002189.3 | NP_002180 |
| IFNα | NM_010502.2 | NP_034632.2. | NM_006900.3. | NP_008831.3 |
| | | | NM_024013.2. | NP076918.1 |
| IFNβ | NM_010510.1 | NP_034640.1 | NM_002176.3. | NP_002167.1 |
| IFNγ | NM_008337.4. | NP_032363.1. | NM_000619.2. | NP_000610.2 |
| IL-2 | NM_008366.3. | NP_032392.1. | NM_000586.3. | NP-000577.2 |
| TGFβ | NM_011577.2. | NP_035707.1 | NM_000660.5. | NP000651.3 |

A “nucleic acid” includes both RNA and DNA. RNA and DNA include, but are not limited to, cDNA, genomic DNA, viral DNA, plasmid DNA, viral RNA, synthetic RNA or DNA, and mRNA. Nucleic acid also includes modified RNA or DNA. In some embodiments, the nucleic acid is a plasmid DNA which constitutes a “vector”. The nucleic acid can be, but is not limited to, a plasmid DNA vector with a eukaryotic promoter which expresses a protein with potential therapeutic action, such as, for example; IFN-α, IL-2, IL-12, or the like.

An “expression vector” refers to an RNA or DNA coding sequence or segment of RNA or DNA that codes for an expression product (e.g., peptide(s) (i.e., polypeptide(s) or protein(s)) or RNA). An expression vector may be, but is not limited to, a virus, an attenuated virus, a plasmid, a linear DNA molecule, or an mRNA. An expression vector is capable of expressing one or more polypeptides in a cell, such a mammalian cell. The expression vector may comprise one or more sequences necessary for expression of the encoded expression product. The expression vector may comprise one or more of an enhancer, a promoter, a terminator, and a polyA signal operably linked to the DNA coding sequence.

The term “plasmid” refers to a nucleic acid that includes at least one sequence encoding a polypeptide (such as any of the described expression vectors) that is capable of being expressed in a mammalian cell. A plasmid can be a closed circular DNA molecule. A variety of sequences can be incorporated into a plasmid to alter expression of the coding sequence are to facilitate replication of the plasmid in a cell. Sequences can be used that influence transcription, stability of a messenger RNA (mRNA), RNA processing, or efficiency of translation. Such sequences include, but are not limited to, 5′ untranslated region (5′ UTR), promoter, introns, and 3′ untranslated region (3′ UTR). Plasmids can be manufactured in large scale quantities and/or in high yield. Plasmids can further be manufacture using cGMP manufacturing. Plasmids can be transformed into bacteria, such as E. coli. The DNA plasmids are can be formulated to be safe and effective for injection into a mammalian subject.

“Operably linked” refers to the juxtaposition of two or more components (e.g., a promoter and another sequence element) such that both components function normally and allow the possibility that at least one of the components can mediate a function that is exerted upon at least one of the other components. For example, a promoter operably linked to a coding sequence will direct RNA polymerase-mediated transcription of the coding sequence into RNA, including mRNA, which may then be spliced (if it contains introns) and, optionally, translated into a protein encoded by the coding sequence. A coding sequence can be “operably linked” to one or more transcriptional or translational control sequences. A terminator/polyA signal operably linked to a gene terminates transcription of the gene into RNA and directs addition of a polyA signal onto the RNA.

A “promoter” is a DNA regulatory region capable of binding an RNA polymerase in a cell (e.g., directly or through other promoter-bound proteins or substances) and initiating transcription of a coding sequence. A promoter may comprise one or more additional regions or elements that influence transcription initiation rate, including, but not limited to, enhancers. A promoter can be, but is not limited to, a constitutively active promoter, a conditional promoter, an inducible promoter, or a cell-type specific promoter. Examples of promoters can be found, for example, in WO 2013/176772. The promoter can be, but is not limited to, CMV promoter, chicken β-actin promoter, modified chicken β-actin promoter (smCBA), opsin promoter, human opsin promoter, truncated human opsin promoter (hOps), rhodopsin kinase promoter, human rhodopsin kinase promoter, Igκ promoter, mPGK, SV40 promoter, β-actin promoter, α-actin promoter, SRα promoter, herpes thymidine kinase promoter, herpes simplex virus (HSV) promoter, mouse mammary tumor virus long terminal repeat (LTR) promoter, adenovirus major late promoter (Ad MLP), rous sarcoma virus (RSV) promoter, and EF1α promoter. The CMV promoter can be, but is not limited to, CMV immediate early promoter, human CMV promoter, mouse CNV promoter, and simian CMV promoter. In some embodiments, the promoter is a retina-specific promoter.

The term “cancer” includes a myriad of diseases generally characterized by inappropriate cellular proliferation, abnormal or excessive cellular proliferation. Examples of cancer include, but are not limited to, breast cancer, triple negative breast cancer, colon cancer, prostate cancer, pancreatic cancer, melanoma, lung cancer, ovarian cancer, kidney cancer, brain cancer, or sarcomas.

Triple negative breast cancer has a negative phenotype for the estrogen (ER) and progesterone (PR) receptors and a lack of gene amplification/protein overexpression for the human epidermal growth factor receptor 2 (HER2). ER/PR negative indicates less than 10% of tumor biopsy cells positively stain for ER or PR.

A “treatment-refractory cancer” is a cancer that does not respond, or has not responded, to at least one prior medical treatment. In some embodiments, a treatment-refractory, with respect to a treatment, indicates an inadequate response to a treatment or the lack of a partial or complete response to the treatment. For example, patients may be considered refractory to a treatment, (e.g., checkpoint inhibitor therapy such as a PD-1 or PD-L1 inhibitor therapy) if they do not show at least a partial response after receiving at least 2 doses of the treatment.

The “tumor microenvironment” refers to the environment around a tumor and includes the non-malignant vascular and stromal tissue that aid in growth and/or survival of a tumor, such as by providing the tumor with oxygen, growth factors, and nutrients, or inhibiting immune response to the tumor. A tumor microenvironment includes the cellular environment in which the tumor exists, including surrounding blood vessels, immune cells, fibroblasts, bone marrow-derived inflammatory cells, lymphocytes, signaling molecules and the extracellular matrix.

The “tumor margin” or “margin tissue” is the visually normal tissue immediately near or surrounding a tumor. Typically, the margin tissue is the visually normal tissue within 0.1-2 cm of the tissue. Tumor margin tissue is often removed when a tumor is surgically resected.

“Treatment” includes, but is not limited to, inhibition or reduction of proliferation of cancer cells, destruction of cancer cells, prevention of proliferation of cancer cells, prevention of initiation of malignant cells, arrest or reversal of the progression of transformed premalignant cells to malignant disease, or amelioration of the disease.

A “pharmacologically effective amount,” “therapeutically effective amount,” “effective amount,” or “effective dose” refers to that amount of an agent to produce the intended pharmacological, therapeutic, or preventive result.

“Subject” refers to an animal, such as a mammal, for example a human. The methods described herein can be useful in both humans and non-human animals. In some embodiments, the subject is a mammal (such as an animal model of disease), and in some embodiments, the subject is human. Veterinary uses are also intended to be encompassed by this invention.

“Electroporation”, “electro-permeabilization,” or “electro-kinetic enhancement” (“EP”) refer to the use of a transmembrane electric field (electroporative) pulse to facilitate entry of biomolecules such as a plasmid, nucleic acid, or drug, into a cell.

“Reversible electroporation” is the reversible, or temporary, permeabilization of cell membranes to molecules that are normally impermeable to the cell membranes using an electric pulse that is below the electric field threshold of the target cells. Because the electric pulse is below the cells' electric threshold, the cells can repair and are not killed by the electric pulse. Reversible electroporation can be used to delivery macromolecules, such as nucleic acid, into a cell without killing the cell. Reversible electroporation is a method that applies electric pulses to facilitate uptake of macromolecules, such as nucleic acids, into cells. Unless indicated otherwise, reference herein to electroporation includes reversible electroporation.

“Intratumoral electroporation” (IT-EP) comprises injecting one or more nucleic acids into a tumor and administering at least one electroporative pulse to the tumor. The one or more nucleic acids can be injected prior to administering the electroporative pulse or substantially simultaneously with administering the electroporative pulse. The electroporative pulse can be performed using any known electroporation device suitable for use in a mammalian subject.

“Locally advanced cancer” is used to describe cancer that has grown outside the organ it started in but has not yet spread to distant parts of the body. Locally advanced cancer may be cancer that has spread to nearby tissue or lymph nodes. Locally advanced cancer is characterized by the most advanced tumors in the absence of distant metastasis. Locally advanced cancer, such as breast cancer, can be either “operable” or “inoperable” based on the probability of achieving negative margins on histopathologic examination after an initial surgical approach that would provide long-term reduction in locoregional recurrence.

“Metastatic cancer” is a cancer that has spread from the part of the body where it started (the primary site) to other parts of the body.

“Operable” describes a cancer that can be treated or removed by surgery.

“Inoperable” describes a cancer that cannot be readily removed surgically, typically because of location or the presence of multiple tumors or metastases.

A “first line therapy” or “first line treatment” is the first or initial treatment, treatment regimen, or regimens given for a given type and stage of cancer. It may be a monotherapy or a set of treatments, including, but not limited to surgery followed by chemotherapy and radiation. It is also called primary treatment or therapy. In some embodiments, first line therapy is administered to a subject as a neoadjuvant prior to surgical resection of a tumor.

A “second line therapy” or “second line treatment” is a second or subsequent treatment, treatment regimen, or regimens for a given type and stage of cancer. It may be a monotherapy or a set of treatments. In some embodiments, second line therapy is administered to a subject after the subject as failed to adequately response to a first line therapy or no longer responds to a first line therapy.

A “neoadjuvant” is a treatment given as a first step to shrink a tumor before the main treatment, which is usually surgery, is given.

### B. Methods for Treating Cancer

Described are methods for treating cancer. The described methods can be used to reduce the size of one or more tumors in a subject, inhibit the growth of cancer cells in a subject, inhibiting or reducing metastases, reduce or inhibit the development of new tumors or metastases in a subject, and/or reduce recurrence of cancer in a subject suffering from cancer.

Therapy is achieved treating a subject with an immunostimulatory cytokine, an immune checkpoint inhibitor, and a chemotherapeutic agent. The immunostimulatory cytokine (e.g., IL-12) is administered to a tumor in the subject by intratumoral electroporation of a nucleic acid expression vector encoding the immunostimulatory cytokine. The immune checkpoint inhibitor and the chemotherapeutic agent can be administered to the subject according the to the product label for the immune checkpoint inhibitor and the chemotherapeutic agent (e.g., systemically. Immune checkpoint inhibitor therapy and chemotherapy may occur before, during, or after intratumoral delivery by electroporation of the expression vector encoding the immunostimulatory cytokine.

In some embodiments, the methods comprise
a) administering at least one immunostimulatory cytokine treatment cycle to the subject, the cycle comprising: administering to the tumor, by IT-EP, a composition comprising a therapeutically effective amount of an expression vector encoding at least one immunostimulatory cytokine.
b) administering at least one immune checkpoint inhibitor treatment cycle to the subject, the cycle comprising: administering to the subject, by intravenous infusion, a composition comprising a therapeutically effective amount of an immune checkpoint inhibitor; and
c) administering at least one chemotherapeutic treatment cycle to the subject, the cycle comprising: administering to the subject a composition comprising a therapeutically effective amount of a chemotherapeutic.
   In some embodiments, the immunostimulatory cytokine comprises IL-12. In some embodiments, the immune checkpoint inhibitor comprises a PD-1/PD-L1 antagonist (e.g., an anti-PD-1 antibody or an anti-PD-L1 antibody). In some embodiments, the chemotherapeutic comprises nab-paclitaxel. In some embodiments, the immunostimulatory cytokine comprises IL-12, the immune checkpoint inhibitor comprises a PD-1/PD-L1 antagonist (e.g., an anti-PD-1 antibody or an anti-PD-L1 antibody) and the chemotherapeutic comprises nab-paclitaxel.

In some embodiments, the methods comprise injecting the cancerous tumor with an effective dose of at least one expression vector coding for at least one immunostimulatory cytokine and administering electroporation therapy to the tumor on days 1, 5, and 8 of a 3-6 week cycle. In some embodiments, the methods comprise injecting the cancerous tumor with an effective dose of at least one expression vector coding for at least one immunostimulatory cytokine and administering electroporation therapy to the tumor on days 1, 5, and 8 of a 6 week cycle. In some embodiments, the methods comprise injecting the cancerous tumor with an effective dose of at least one expression vector coding for at least one immunostimulatory cytokine and administering electroporation therapy to the tumor on days 1, 5, and 8 of a 6 week cycle for at least 1 cycle. In some embodiments, the methods comprise injecting the cancerous tumor with an effective dose of at least one expression vector coding for at least one immunostimulatory cytokine and administering electroporation therapy to the tumor on days 1, 5, and 8 of a 6 week cycle for at least 2 cycles. In some embodiments, the methods comprise injecting the cancerous tumor with an effective dose of at least one expression vector coding for at least one immunostimulatory cytokine and administering electroporation therapy to the tumor on days 1, 5, and 8 every 6 weeks for at least 2 cycles. In some embodiments, the methods comprise injecting the cancerous tumor with an effective dose of at least one expression vector coding for at least one immunostimulatory cytokine and administering electroporation therapy to the tumor on days 1, 5, and 8 every 6 weeks for up to 17 cycles or more. In some embodiments, the methods comprise injecting the cancerous tumor with an effective dose of at least one expression vector coding for at least one immunostimulatory cytokine and administering electroporation therapy to the tumor on days 1, 5, and 8 every 6 weeks for up to 2 years or more. For each administration, the administration can occur on the indicated day ±2 days; e.g., day 1±2 days, day 5±2 days, day 8±2 days.

In some embodiments, the methods comprise administering an effective dose of an immune checkpoint inhibitor to the subject on day 1 of a 3 week cycle. In some embodiments, the methods comprise administering an effective dose of an immune checkpoint inhibitor to the subject on day 1 of a 3 week cycle for at least one cycle. In some embodiments, the methods comprise administering an effective dose of an immune checkpoint inhibitor to the subject on day 1 of a 3 week cycle for at least 2 cycles. In some embodiments, the methods comprise administering an effective dose of an immune checkpoint inhibitor to the subject on day 1 every 3 weeks for at least 2 cycles. In some embodiments, the methods comprise administering an effective dose of an immune checkpoint inhibitor to the subject on day 1 every 3 weeks for up to 33 cycles or more. In some embodiments, the methods comprise administering an effective dose of an immune checkpoint inhibitor to the subject on day 1 every 3 weeks for up to 2 years or more. For each administration, the administration can occur on the indicated day ±2 days; e.g., day 1±2 days.

In some embodiments, the methods comprise administering an effective dose of a chemotherapeutic agent to the subject on days 1, 8, and 15 of a 4 week cycle. In some embodiments, the methods comprise administering an effective dose of a chemotherapeutic agent to the subject on days 1, 8, and 15 of a 4 week cycle for at least 1 cycle. In some embodiments, the methods comprise administering an effective dose of a chemotherapeutic agent to the subject on days 1, 8, and 15 of a 4 week cycle for at least 2 cycles. In some embodiments, the methods comprise administering an effective dose of a chemotherapeutic agent to the subject on days 1, 8, and 15 every 4 weeks for at least 2 cycles. In some embodiments, the methods comprise administering an effective dose of a chemotherapeutic agent to the subject on days 1, 8, and 15 every 4 weeks cycle for up to 25 cycles or more. In some embodiments, the methods comprise administering an effective dose of a chemotherapeutic agent to the subject on days 1, 8, and 15 every 4 weeks for up to 2 years or more. For each administration, the administration can occur on the indicated day ±2 days; e.g., day 1±2 days, day 8±2 days, day 15±2 days.

In some embodiments, the immunostimulatory cytokine, the immune checkpoint inhibitor, and the chemotherapeutic agent are administered to the patient on the same day of the initial cycle; i.e., day 1 of the first cycle is the same day for each cycle.

In some embodiments, immunostimulatory cytokine, the checkpoint inhibitor and the chemotherapeutic agent are administered to the subject on day 1. The immunostimulatory cytokine is then administered again on day 5 and day 8, and chemotherapeutic agent is administered again on day 8 and day 15. The immunostimulatory cytokine is administered every 6 weeks on days 1, 5, and 8; the checkpoint inhibitor is administered every 3 weeks on day 1; and the chemotherapeutic agent is administered every 4 weeks on days 1, 8, and 15.

In some embodiments, the immunostimulatory cytokine is administered every 6 weeks on days 1, 5, and 8; the checkpoint inhibitor is administered every 3 weeks on day 1; and the chemotherapeutic agent is administered according to the standard of care for the chemotherapeutic agent.

The described methods are contemplated for use in numerous types of malignant tumors (i.e., cancer). For example, the devices and methods described herein are contemplated for use in adrenal cortical cancer, anal cancer, bile duct cancer (e.g., periphilar cancer, distal bile duct cancer, intrahepatic bile duct cancer) bladder cancer, benign and cancerous bone cancer (e.g., osteoma, osteoid osteoma, osteoblastoma, osteochrondroma, hemangioma, chondromyxoid fibroma, osteosarcoma, chondrosarcoma, fibrosarcoma, malignant fibrous histiocytoma, giant cell tumor of the bone, chordoma, lymphoma, multiple myeloma), brain and central nervous system cancer (e.g., meningioma, astocytoma, oligodendrogliomas, ependymoma, gliomas, medulloblastoma, ganglioglioma, Schwannoma, germinoma, craniopharyngioma), breast cancer (e.g., ductal carcinoma in situ, infiltrating ductal carcinoma, infiltrating lobular carcinoma, lobular carcinoma in situ, gynecomastia), Castleman disease (e.g., giant lymph node hyperplasia, angiofollicular lymph node hyperplasia), cervical cancer, colorectal cancer, endometrial cancer (e.g., endometrial adenocarcinoma, adenocanthoma, papillary serous adenocarcinoma, clear cell) esophagus cancer, gallbladder cancer (mucinous adenocarcinoma, small cell carcinoma), gastrointestinal carcinoid tumors (e.g., choriocarcinoma, chorioadenoma Destruens), Hodgkin's disease, non-Hodgkin's lymphoma, Kaposi's sarcoma, kidney cancer (e.g., renal cell cancer), laryngeal and hypopharyngeal cancer, liver cancer (e.g., hemangioma, hepatic adenoma, focal nodular hyperplasia, hepatocellular carcinoma), lung cancer (e.g., small cell lung cancer, non-small cell lung cancer), mesothelioma, plasmacytoma, nasal cavity and paranasal sinus cancer (e.g., esthesioneuroblastoma, midline granuloma), nasopharyngeal cancer, neuroblastoma, oral cavity and oropharyngeal cancer, ovarian cancer, pancreatic cancer, penile cancer, pituitary cancer, prostate cancer, retinoblastoma, rhabdomyosarcoma (e.g., embryonal rhabdomyosarcoma, alveolar rhabdomyosarcoma, pleomorphic rhabdomyosarcoma), salivary gland cancer, skin cancer, both melanoma and non-melanoma skin cancer), stomach cancer, testicular cancer (e.g., seminoma, nonseminoma germ cell cancer), thymus cancer, thyroid cancer (e.g., follicular carcinoma, anaplastic carcinoma, poorly differentiated carcinoma, medullary thyroid carcinoma, thyroid lymphoma), vaginal cancer, vulvar cancer, and uterine cancer (e.g., uterine leiomyosarcoma). In some embodiments, the methods are contemplated for use in treating triple negative breast cancer. In some embodiments, the methods are contemplated for use in treating melanoma.

The treated tumor can be a cutaneous tumor, a subcutaneous tumor, or a visceral tumor. In some embodiments, the described methods and expression vectors can be used to treat primary tumors as well as distant (i.e., untreated) tumors and metastases.

The cancer can be stage 0 (in situ), stage I, stage II, stage III, or stage IV. The cancer can be operable or inoperable. The cancer can be locally advanced cancer (stage II or III) or metastatic (stage IV). The cancer can be, but is not limited to, breast cancer, triple negative breast cancer, melanoma, head and neck cancer, squamous cell carcinoma, basal cell carcinoma, and Merkel cell carcinoma. In some embodiments, the cancer is operable. In some embodiments, the cancer in inoperable. In some embodiments, the cancer in inoperable locally advanced or metastatic cancer.

Stage 0 (in situ) cancers are still located in the place they started (in situ) and have not spread to nearby tissues.

Stage I cancers are usually a small cancer or tumor that has not grown deeply into nearby tissues. It also has not spread to the lymph nodes or other parts of the body. It is often called early-stage cancer.

Stage II cancers are larger than stage I, but haven't spread to other locations.

Stage III cancers are larger cancers or tumors that have grown more deeply into nearby tissue. They may have also spread to lymph nodes but not to other parts of the body.

Stage IV cancers have spread beyond the original tumor to other organs or parts of the body. It may also be called advanced or metastatic cancer.

Cancers may also be staged according to the TNM system according to the size and extent of the main (original) tumor (T), the number of lymph nodes to which the cancer has spread (N), and the degree of metastasis (M). Each of T, N, and M may be assigned a number: T0-T6, N0-Nx (e.g., x=1, 2, 3), and MO-Ml. Tx, Nx, or Mx indicates the tumor or metastasis cannot be measured.

Described are methods for treatment of a tumor in a subject comprising, administering to the subject an effective dose of an expression vector encoding an immunostimulatory cytokine (e.g., IL-12). The expression vector encoding the immunostimulatory cytokine is administered to the subject by injecting the expression vector into the tumor, tumor microenvironment, and/or tumor margin tissue and administering electroporation therapy to the tumor, tumor microenvironment, and/or the tumor margin tissue (IT-EP treatment).

IT-EP immunostimulatory cytokine therapy or treatment comprises injecting a tumor, tumor microenvironment, and/or tumor margin tissue with an effective dose of an expression vector encoding an immunostimulatory cytokine and administering at least one electroporative pulse to the tumor. Electroporation is administered within 10 minutes, within 8 minutes, within 5 minutes, within 4 minutes, within 3 minutes, within 2 minutes, or within 1 minute of IL-12 plasmid injection. The electroporative pulse can be performed using any known electroporation device suitable for use in a mammalian subject. In some embodiments, the electroporation device contains six needles in an about 0.5 to about 1.0 cm diameter circular configuration. The electroporation device needles are placed into or around the sites where plasmid was injected. IT-EP immunostimulatory cytokine therapy results in localized expression of the immunostimulatory cytokine in the tumor microenvironment.

In some embodiments, each individual tumor is injected and electroporated before injecting and electroporating the next tumor. If lesions are in close proximity, these lesions can be injected first and then undergo EP. In some embodiments, all tumors in a subject are injected followed by electroporation of the injected tumors.

IT-EP IL-12 therapy or treatment comprises injecting a tumor, tumor microenvironment, and/or tumor margin tissue with an effective dose of an expression vector encoding IL-12 and administering at least one electroporative pulse to the tumor. The electroporative pulse can be performed using any known electroporation device suitable for use in a mammalian subject. IT-EP IL-12 results in localized expression of IL-12 in the tumor microenvironment.

IT-EP IL-12 in TNBC lesions is expected to increase infiltration of T cells (TILs), such as CD8+ T cells to the tumor. Increased TILs within the tumor microenvironment has been associated with improved disease-free and overall survival. Expression of IL-12 in the tumor also increases cell-mediated immune response to the tumor such as through activation of natural killer cells and cytotoxic TILs, inhibition of regulatory T cells and myeloid-derived suppressor cells. (Kobayashi M L et al. (1989). “Identification and purification of natural killer cell stimulatory factor (NKSF), a cytokine with multiple biologic effects on human lymphocytes.” The Journal of experimental medicine 170(3): 827-845; Brunda M J et al. (1993). “Antitumor and antimetastatic activity of interleukin 12 against murine tumors.” The Journal of experimental medicine 178(4): 1223-1230; Steding C E et al. (2011) “The role of interleukin-12 on modulating myeloid-derived suppressor cells, increasing overall survival and reducing metastasis.” Immunology 133(2): 221-238), induction of interferon-gamma (IFNγ) production, and upregulation of antigen processing and presentation machinery within tumors. Local expression of IL-12 does not lead to high levels of systemic IL-12, thus avoiding the toxicity associated with systemic administration of IL-12. IT-EP IL-12 has been shown to enhance clinical response to checkpoint inhibitor therapy in treating cancer.

IL-12 is a heterodimeric cytokine having both IL-12A (p35) and IL-12B (p40) subunits. A expression vector encoding IL-12 can comprise a nucleic acid sequence encoding an IL-12 p40-IL-12 p35 fusion protein (an IL-12 p70), a nucleic acid sequence encoding an IL-12 p35-IL-12 p40 fusion protein (an IL-12 p70), or an IL-12 p35 subunit and an IL-12 p40 subunit. The nucleic acid sequences encoding the IL-12 p35 and IL-12 p40 subunits can be on a contiguous nucleic acid sequence separated by a translation modification element, allowing both subunits to be expressed from a single promoter or on a single mRNA. The translation modification element can be an internal ribosome entry site (IRES) element or a ribosome skipping modulator. A ribosome skipping modulator can be, but is not limited to, a 2A element (also termed 2A peptide or 2A self-cleaving peptide). The 2A element can be, but is not limited to, a P2A, T2A, E2A or F2A element. The IL-12 p35 and p40 coding sequences can be expressed from a multicistronic expression vector from a single promoter and separated by an IRES or 2A element.

An expression vector or plasmid may contain a multicistronic expression vector. Multicistronic expression vectors express two or more separate proteins from the same mRNA and contain one or more translation modification elements. In some embodiments, an expression vector encoding IL-12 expresses two or three polypeptides expressed from a single promoter, with one or more translation modification elements to allow the two or three polypeptides to be expressed from a single mRNA. In some embodiments, the expression vector comprises:
(c) P-A-T¹-A′
(a) P-A-T¹-B,
(b) P-B-T¹-A,
(d) P-A-T¹-A′-T²-B or
(e) P-B-T¹-A-T²-A′
   wherein P is a promoter, A and A′ encode IL-12 or IL-12 subunits, T¹ and T² are both translation modification elements, and B encodes a second therapeutic polypeptide. The second therapeutic polypeptide can be, but is not limited to, a checkpoint inhibitor, a genetic adjuvant, a tumor antigen, an anti-CD3 antibody or fragment thereof, a second immunostimulatory cytokine (e.g., CXCL9), or a combination thereof. In some embodiments, T¹ and T² are translation modification elements, independently internal ribosome entry site (IRES) elements or ribosomal skipping modulators. Translation modification elements enable translation of two or more genes from a single transcript. IRES elements provide for initiation of translation from an internal region of an mRNA. Ribosomal skipping modulators allow the ribosome to skip the synthesis of a peptide bond at the C-terminus of the element. A ribosome skipping modulator can be, but is not limited to, a 2A element (also termed 2A peptide or 2A self-cleaving peptide).

The 2A element can be, but is not limited to, a P2A, T2A, E2A or F2A element. Incorporation of translation modulating element results in co-expression of two or more polypeptide from a single polycistronic mRNA.

In some embodiments, the IL-12 expression vector construct comprises the formula represented by: P-A-T-A′ where a) P is an expression promoter; b) A, and A′ encode subunits of an IL-12; and c) T is a translation modification element. In certain embodiments, P is selected from group consisting of human CMV promoter, a simian CMV promoter, SV-40, mPGK, and β-Actin; A encoded IL-12p35, A′ encoded IL-12p40, and T is selected from the group consisting of a P2A and IRES.

The expression vectors can be formulated for in vivo administration. In some embodiments, the expression vectors are formulated for in vivo administration by electroporation. In some embodiments, the expression vectors are formulated for intratumoral administration by electroporation (IT-EP). The nucleic acids can be made using methods known in the art.

In some embodiments, a cycle of IT-EP IL-12 therapy comprises IT-EP administration of a nucleic acid encoding an immune stimulator on day 1 (±2 days); days 1 (±2 days) and 5 (±2 days); days 1 (±2 days) and 8 (±2 days); or days 1 (±2 days), 5 (±2 days), and 8 (±2 days) or days 1 (±2 days), 5 (±2 days), and 8 (±2 days) of a 4 or 6 week cycle. In some embodiments the cycle is a 6 week cycle.

Immune checkpoint inhibitors may be in the form of antibodies, antigen-binding fragments, or nanobodies, each of which can be encoded in an expression vector and delivered to the tumor by electroporation, or delivered as proteins/peptides. Proteins/peptides can be administered systemically. Administration of the immune checkpoint inhibitor therapeutic can occur before, during or after intratumoral delivery by electroporation of an immunostimulatory cytokine, e.g., IL-12.

Antibodies exist as full length intact antibodies or as a number of well-characterized fragments. Antibody fragments include, but are not limited to F(ab), F(ab′), F(ab′)2, and scFv (single chain variable fragment). A variety of antibody fragments may be synthesized de novo either chemically or by utilizing recombinant DNA methodology.

Other immune checkpoint inhibitors include, but are not limited to, soluble antagonists, such as an extracellular domain of a checkpoint protein. A soluble immune checkpoint inhibitor can be encoded in an expression vector and delivered to the tumor by electroporation, or delivered as proteins/peptides systemically.

In some embodiments, administering at least one immune checkpoint inhibitor treatment cycle comprises administering the effective dose of the immune checkpoint inhibitor, day 1 (±2 days) of a 3-6 week cycle. In some embodiments, the immune checkpoint inhibitor is administered on day 1 (±2 days) of a 3 week cycle. In some embodiments, the immune checkpoint inhibitor is administered on day 1 (±2 days) of a 4 week cycle. In some embodiments, the immune checkpoint inhibitor comprises an anti-PD-1 antibody or anti-PD-L1 antibody or antigen binding fragment thereof.

Pembrolizumab is a humanized immunoglobulin G4 (IgG4) monoclonal antibody (mAb) with specificity of binding to the PD-1 receptor, thus inhibiting its interaction with PD-L1 and programmed death ligand 2 (PD-L2). Pembrolizumab is indicated for the treatment of patients across a number of indications including unresectable and/or metastatic melanoma and non-small cell lung cancer (NSCLC). Pembrolizumab is administered according to the drug product label. In some embodiments, pembrolizumab is administered IV at the dose of about 200 mg using a 30-minute (−5/+10 minutes) IV infusion on Day 1 (±2 days) of each 3-week cycle. On any give cycle, one or more of the injections may be withheld or administered at a different dose or on a different day as medically necessitated. Altering dosage (administering at a different dose) can comprise discontinuing an infusion, altering the induction rate, pausing and restarting infusion, or administering a difference amount of drug during an infusion. Adverse reactions to pembrolizumab may also be treated according to the manufacture's recommendations (e.g., drug product label).

Expression of Programmed death-ligand 1 (PD-L1) in tumors leads in inhibition of the antitumor activity of the TILs. Therefore, administration of anti-PD-1/anti-PD-L1 therapy is expected to reverse the immune inhibition mediated by PD-L1 expression. In some embodiments, subjects are screened for expression of PD-L1 in tumor samples. Subjects expressing PD-L1 are expected to have increased response to of anti-PD-1/anti-PD-L1 therapy. Therapeutic studies in mouse models have shown that administration of antibodies blocking PD-1/PD-L1 interaction enhances infiltration of tumor-specific CD8+ T cells.

Any of the checkpoint inhibitors described herein may be administered to the subject.

Administering at least one chemotherapeutic treatment cycle comprises administering the chemotherapeutic according to generally accepted practices (i.e., according to the product label or generally accepted standard of care or other recommended dose level) for the chemotherapeutic in treating the particular type and stage of cancer to be treated. In some embodiments, the chemotherapeutic agent is administered according to its recognized administration route and/or dosages levels. Clinically relevant doses of chemotherapeutic are used when applicable. For example, with some drugs, the chemotherapeutic is given only on the first day of a cycle. Other chemotherapeutics are given for a few days in a row, or once a week. At the end of a cycle, the chemotherapeutic treatment repeats to start the next cycle. The chemotherapeutic can be administered, for example, by intravenous injection, intravenous infusion, intramuscular injection, oral administration (such as by pill or capsule or liquid).

In cases where the cancer is TNBC, the chemotherapeutic can be, but is not limited to, an anthracycline (e.g., daunorubicin, doxorubicin, pegylated liposomal doxorubicin, epirubicin), a cyclophosphamide, an alkylating agent (e.g., thiotepa), a taxane (e.g., docetaxel, paclitaxel, nab-paclitaxel), a nucleotide analog or antimetabolite (e.g., fluorouracil (5-FU), gemcitabine, methotrexate, capecitabine), a microtubule inhibitor (e.g., eribulin), platinum agent (e.g., cisplatin, carboplatin), a PI3K inhibitor (e.g., alpelisib), a poly ADP-ribose polymerase (PARP) inhibitor (e.g., olaparib, talazoparib), cytoxan, ixabepilone, mutanycin, vinorelbine, or combinations thereof. In some embodiments, the cancer is breast cancer or TNBC and the chemotherapeutic agent comprises a taxane such as paclitaxel or nab-paclitaxel.

In some embodiments the chemotherapeutic is nab-paclitaxel the nab-paclitaxel is administered according to standard of care for dosing regimen and toxicity management. In some embodiments, the nab-paclitaxel is administered to the subject on days 1 (±2 days), 8 (±2 days), and 15 (±2 days) of a 4 week cycle. “Nab-paclitaxel” is protein-bound paclitaxel, also known as nanoparticle albumin-bound paclitaxel (Abraxane), that is an injectable formulation of paclitaxel used to treat breast cancer, lung cancer and pancreatic cancer, among others. Paclitaxel kills cancer cells by preventing the normal breakdown of microtubules during cell division. Nab-paclitaxel is paclitaxel is bonded to albumin as a delivery vehicle. Nab-paclitaxel can be administered at a dose of about 100 mg/m² to about 260 mg/m² on days 1, 8, and 15 (each ±2 days) every 4 weeks, intravenously over 30 minutes (−5/+10 minutes). In some embodiments, the nab-paclitaxel is administered according the drug product label. On any give cycle, one or more of the injections may be altered withheld or administered at a different dose or on a different day as medically necessitated. Altering dosage (administering at a different dose) can comprise discontinuing an infusion, altering the induction rate, pausing and restarting infusion, or administering a difference amount of drug during an infusion. Adverse reactions to nab-paclitaxel may also be treated according to the manufacture's recommendations (e.g., drug product label).

In some embodiments, the chemotherapeutic is paclitaxel and is administered to the subject as a dose or 120 mg/m² per week, or 175 mg/m² on day one every three weeks.

In some embodiments, combinations of chemotherapeutic agents can be used. Different chemotherapeutic agents can be used in the same cycle or in different cycles.

Described are methods of treating cancer in a subject comprising administering concurrent cycles of IT-EP IL-12 therapy, immune checkpoint inhibitor therapy, and chemotherapeutic agent therapy. IT-PE IL-12 therapy comprises injecting one or more tumors in the subject with an effective does of an expression vector encoding IL-12 and administering electroporation therapy to the tumor. A cycle of IT-EP IL-12 therapy comprises administering IT-EP IL-12 on day 1±2 days, day 5±2 days, and day 8±2 days in a 6 week cycle. A cycle of immune checkpoint inhibitor therapy comprises administering an effective dose of an anti-PD-1 antibody or anti-PD-L1 antibody on day 1±2 days of a 3 week cycle. A cycle of chemotherapeutic agent therapy comprises administering an effective dose of nab-paclitaxel or paclitaxel on day 1±2 days, day 8±2 days, and day 15±2 days in a 4 week cycle. Treatment of a subject may commence on day 1, with each of IT-EP IL-12 therapy, immune checkpoint inhibitor therapy, and chemotherapeutic agent therapy proceeding according to its cycle schedule.

Described are methods of treating cancer in a subject comprising administering concurrent cycles of IT-EP IL-12 therapy, anti-PD-1 or anti-PD-L1 (anti PD-1/PD-L1) therapy, and nab-paclitaxel or paclitaxel therapy. IT-PE IL-12 therapy comprises injecting one or more tumors in the subject with an effective does of an expression vector encoding IL-12 and administering electroporation therapy to the tumor. A cycle of IT-EP IL-12 therapy comprises administering IT-EP IL-12 on day 1±2 days, day 5±2 days, and day 8±2 days in a 6 week cycle. A cycle of anti-PD-1/PD-L1 therapy comprises administering an effective dose of an anti-PD-1 antibody or anti-PD-L1 antibody on day 1±2 days of a 3 week cycle. The anti-PD-1 antibody or anti-PD-L1 antibody may be selected from the group consisting of: nivolumab, pembrolizumab, pidilizumab, durvalumab, atezolizumab, avelumab, cemiplimab, sintilimab, toripalimab, or camrelizumab. A cycle of nab-paclitaxel/paclitaxel therapy comprises administering an effective dose of nab-paclitaxel or paclitaxel on day 1±2 days, day 8±2 days, and day 15±2 days in a 4 week cycle. Treatment of a subject may commence on day 1, with each of IT-EP IL-12 therapy, anti-PD-1/PD-L1 therapy, and paclitaxel/paclitaxel therapy proceeding according to its cycle schedule.

One days in which pembrolizumab and IT-EP IL-12 are administered on the same day, pembrolizumab may be administered prior to, concurrently with, or after administration of IT-EP IL-12. In some embodiments, IT-EP IL-12 is administered prior to administration of pembrolizumab. One days in which nab-paclitaxel and IT-EP IL-12 are administered on the same day, nab-paclitaxel may be administered prior to, concurrently with, or after administration of IT-EP IL-12. One days in which pembrolizumab and nab-paclitaxel are administered on the same day, pembrolizumab may be administered prior to, concurrently with, or after administration of nab-paclitaxel.

Clinical outcomes in cancer trials may be measured by the Response Evaluation Criteria In Solid Tumors (RECIST) criteria. RECIST provides guidelines that define when tumors improve (“respond”), stay the same (“stabilize”), or worsen (“progress”).

A sum of the longest diameter (LD) for all target lesions is used as the baseline sum LD and used as reference by which to characterize the objective tumor response in the targeted lesion(s).
a. Complete Response (CR) is the disappearance of all target lesions;
b. Partial Response (PR) shows at least a 30% decrease in the sum of the LD of target lesions;
c. Stable Disease (SD) occurs if there no sufficient shrinkage to qualify for PR nor sufficient increase to qualify for Progressive Disease (PD); and
d. Progressive Disease (PD): At least a 20% increase in the sum of the LD of target lesions or the appearance of one or more new lesions.

### C. Formulation

Any of the described expression vectors, checkpoint inhibitors, or chemotherapeutics may comprise one or more pharmaceutically acceptable excipients. In some embodiments, one or more pharmaceutically acceptable excipients (including vehicles, carriers, diluents, and/or delivery polymers) are added to the expression vector encoding the immunostimulatory cytokine, checkpoint inhibitor, or chemotherapeutic.

Pharmaceutically acceptable excipients (“excipients”) are substances other than the Active Pharmaceutical ingredient (API, therapeutic product; e.g., nucleic acid encoding a coronavirus antigenic polypeptide or immune stimulator) that are intentionally included in the drug delivery system. Excipients do not exert or are not intended to exert a therapeutic effect at the intended dosage. Excipients may act to a) aid in processing of the drug delivery system during manufacture, b) protect, support or enhance stability, bioavailability or patient acceptability of the API, c) assist in product identification, and/or d) enhance any other attribute of the overall safety, effectiveness, of delivery of the API during storage or use. A pharmaceutically acceptable excipient may or may not be an inert substance.

Excipients include, but are not limited to: agents that enhance transfection, absorption enhancers, anti-adherents, anti-foaming agents, anti-oxidants, binders, buffering agents, carriers, coating agents, colors, delivery enhancers, delivery polymers, dextran, dextrose, diluents, disintegrants, emulsifiers, extenders, fillers, flavors, glidants, humectants, lubricants, oils, polymers, preservatives, saline, salts, solvents, sugars, suspending agents, sustained release matrices, thickening agents, tonicity agents, vehicles, water-repelling agents, and wetting agents. Agents that enhance transfection include, but are not limited to, lipids, cationic lipids, lipids, polycations, cell-penetrating peptides, and combinations thereof.

The expression vectors, checkpoint inhibitors, or chemotherapeutics can contain other additional components commonly found in pharmaceutical compositions. Such additional components can include, but are not limited to, anti-pruritics, astringents, local anesthetics, or anti-inflammatory agents (e.g., antihistamine, diphenhydramine, etc.). As used herein, “pharmacologically effective amount,” “therapeutically effective amount,” or simply “effective dose” refers to that amount of a described nucleic acid to produce the intended pharmacological, therapeutic, or preventive result.

### D. Electroporation

The described expression vectors encoding the immunostimulatory cytokine can be delivered by electroporation. Electroporation comprises administering at least one electroporative pulse to a cell, tissue, or tumor. Electroporation (EP) is a technique that applies electric pulses to transiently permeabilize a cell membrane, promoting uptake of macromolecules such as nucleic acids into the cell. In vivo EP has been used in several clinical trials to deliver DNA vaccines and drugs to various tissues (Draghia-Akli R et al. “Gene and cell therapy: Therapeutic mechanisms and strategies.” 2009). Electroporation has been shown to dramatically improve gene delivery (100-1000-fold; Sardesai et al. “Electroporation Delivery of DNA Vaccines: Prospects for Success.” Curr Opin Immunol 2011 June, 23(3):421-429; Livingston B D et al. “Comparative performance of a licensed anthrax vaccine versus electroporation based delivery of a PA encoding DNA vaccine in rhesus macaques.” Vaccine, 2010 28(4):1056-61). In vivo electroporation is a gene delivery technique that has been used successfully for efficient delivery of plasmid DNA to many different tissues. Use of in vivo electroporation enhances plasmid DNA uptake in tumor tissue, resulting in expression within the tumor. The described expression vectors can be administered to a subject before, during, or after administration of the electric pulse. The expression vector can be administered at or near the tumor in a subject. The described expression vectors can be injected into a tumor using a hypodermic needle.

Electroporation can be performed using any known electroporation device suitable for use in a mammalian subject. Electroporation devices suitable for use with the described compounds, compositions, and methods include, but are not limited to, those described in U.S. Pat. Nos. 7,245,963, 5,439,440, 6,055,453, 6,009,347, 9,020,605, and 9,037,230, and U.S. Patent Publication Nos. 2005/0052630, 2019/0117964, 2020/0246612 and patent applications PCT/US2019/030437, each of which in incorporated herein by reference.

In some embodiments, electroporation comprises the administration of one or more voltage pulses. The nature of the electric field to be generated is determined by the nature of the tissue, the size of the selected tissue and its location. The voltage pulse that can be delivered to the tumor may be about 300 V/cm to about 1500V/cm. In some embodiments, the voltage pulse is about 700 V/cm to 1500 V/cm. In some embodiments, the voltage pulse is about 1300 V/cm to 1500 V/cm. In some embodiments, the voltage pulse is about 1500 V/cm. In some embodiments, the voltage pulse may be about 600 V/cm, 650 V/cm, 700 V/cm, 750 V/cm, 800 V/cm, 850 V/cm, 900 V/cm, 950 V/cm, 1000 V/cm, 1050 V/cm, 1100 V/cm, 1150 V/cm, 1200 V/cm, 1250 V/cm, 1300 V/cm, 1350 V/cm, 1400 V/cm, 1450 V/cm, or 1500 V/cm. In some embodiments, the voltage pulse is about 300 V/cm to 700 V/cm. In some embodiments, the electric is about 100 V/cm, 150 V/cm, 200 V/cm, 250 V/cm, 300 V/cm, 350 V/cm, or 400 V/cm, 450 V/cm, 500 V/cm, 550 V/cm, 600 V/cm 650 V/cm, or 700 V/cm.

The pulse duration of the electroporative pulse may be from about 10 μsec to about 1 second. In some embodiments, the pulse duration is from about 10 μsec to about 100 milliseconds (ms). In some embodiments, the pulse duration is 100 μsec, 1 ms, 10 ms, or 100 ms. The interval between pulses sets can be any desired time, such as one second. The waveform, electric field strength and pulse duration may also depend upon the type of cells and the type of molecules that are to enter the cells via electroporation.

The waveform of the electrical signal provided by the pulse generator can be an exponentially decaying pulse, a square pulse, a unipolar oscillating pulse train, a bipolar oscillating pulse train, or a combination of any of these forms. Square wave electroporation systems deliver controlled electric pulses that rise quickly to a set voltage, stay at that level for a set length of time (pulse length), and then quickly drop to zero.

1 to 100 pulses may be administered. In some embodiments, 1, 2, 3, 4, 5, 6, 7, 8, 9, or 10 pulses are administered. In some embodiments, 6 pulses are administered. In some embodiments, 6 ×0.1 msec pulses are administered. In some embodiments, 6 pulses are administered. In some embodiments, 6 ×0.1 msec pulses are administered at 1300-1500 V/cm. In some embodiments 8 pulses are administered. In some embodiments 8 ×10 msec pulses are administered. In some embodiments 8 ×10 msec pulses are administered at 300-500 V/cm.

On any give cycle, one or more of the injections may be withheld or administered at a different dose or on a different day as medically necessitated.

The electroporation device can comprise a single needle electrode, a pair of needle electrode, or a plurality or array of needle electrodes. In some embodiments, the electroporation device an comprise a hypodermic needle or equivalent. In some embodiments, the electroporation device can comprise an electro-kinetic device (“EKD device”) able to produce a series of programmable constant-current pulse patterns between electrodes in an array based on user control and input of the pulse parameters. In some embodiments, the EP applicator is inserted such that the electrodes span the nucleic acid injection site.

In some embodiments, the electroporation (EP) device system consists of 3 main components:
(a) an Electroporation Generator that generates electric pulses,
(b) a sterile Applicator Tip containing a needle array, and
(c) an Applicator Handle that connects to the Electroporation Generator at the proximal end and connects to the Applicator Tip at the distal end.

An applicator tip can contain 1, 2, 3, 4, 5, 6, 7, 8, 9, 10, or more needles (electrodes). In some embodiments, the applicator tips contains 6 needles in a circular array with a circle diameter of 0.5-1 cm. The needle length can be 0.5-2 cm. The applicator tip can contain needles of variable insertable length, such that they can be adjusted for an insertion depth of 0-2 cm. In some embodiments, the needles are 1.5 cm in length and can be adjusted for an insertion depth of 0.1-1.5 cm.

The electroporation device can be triggered (activated) to deliver the electroporation pulse(s) by means of a foot switch. The electroporation generator can deliver controlled electrical pulses in a square wave pulse pattern. For a six needle applicator tip, the electroporation pulses occur between opposing needles relative to the circular arrangement. The electroporation device can administer a first pulse between a first pair of opposing needles followed by a second pulse of opposite polarity between the same pair of opposing needles. The electroporation device can then deliver a third pulse between a second pair of opposing needles (the second pair being different than the first pair) and a fourth pulse of opposite polarity between the second pair of needles. Two pulses, one of each polarity, are administered between pair of opposing needled until two pulses have been delivered to all pairs of needles. Thus, for a six needle applicator tip, two pulses are delivered to each pair of opposing electrodes for a total of six pulses. For a two needle electrode, pulses or opposite polarity are delivered until the desired number of pulsed is reached. In some embodiments, any number of pulses may be used in a treatment. In some embodiments, 6 pulses are used. In some embodiments, 8 pulses are used. In some embodiments, 10 pulses are used.

### E. Kits

Any of the described expression vectors, checkpoint inhibitors, and/or chemotherapeutics may be packaged or included in a kit, container, pack, or dispenser. The kit, containers, pack, or dispensers can contain a sufficient amount of expression vectors, checkpoint inhibitors, and/or chemotherapeutics to provide a single effective dose or multiple effective doses. Any of the described expression vectors, checkpoint inhibitors, and/or chemotherapeutics may be packaged in pre-filled syringes or vials. The expression vectors, checkpoint inhibitors, and/or chemotherapeutics may be provided as a lyophilized powder or they may be provided in a solution. A kit can comprise a reagent utilized in performing a method disclosed herein. A kit can also comprise an electroporation applicator. In some embodiments, the kit comprises an expression vector encoding an immunostimulatory cytokine and an electroporation device or applicator. In some embodiments, the kit comprises one or more or the described expression vector, one or more electroporation applicators, syringes, and injection needles. In some embodiments, a kit further contains one or more of: instructions for use, or a notice in a form prescribed by a governmental agency regulating the manufacture, use or sale of the products.

### EXAMPLES

### Example 1. Plasmids Encoding IL-12

A. IL-12: DNA plasmid vector (pUMVC3-hIL-12-NGVL331, referred to as “pIL-12,” “IL-12 plasmid,” “TAVO,” or “Tavokinogene Telseplasmid”), expressing IL-12 cDNA, contained the human IL-12 p35 and p40 subunits separated by an internal ribosomal entry site driven by a single CMV promoter. pIL-12 was formulated in phosphate buffered saline (PBS) for direct intratumoral injection followed by in vivo EP. GMP-grade pIL-12 was manufactured by VGXI USA and available batches were supplied as 2.0 ml vials at a concentration of 0.5 mg/ml and fill volume of 1.5 ml. Unopened vials of pIL-12 were stored in a secure, continuously temperature monitored and alarmed freezer in the pharmacy or other appropriate secure location at −20° C.±5° C.

B. IL-12 P2A: A pUMVC3 backbone was purchased from Aldevron (Fargo, ND). A 1071 bp DNA fragment (gene block) encoding the translation modulating element P2A linked in-frame to hIL12p40 (P2A-hIL12p40) was purchased from IDT (Coralville, IA). The p40 geneblock was PCR amplified using Phusion polymerase (NEB, Ipswich MA, cat. #M0530S) and ligated into pUMVC3 downstream of the CMV promoter/enhancer using standard restriction enzyme pairing and T4 DNA ligase (Life Technologies, Grand Island NY, cat. #15224-017). Positives clones of P2A-hIL12p40/pOMI2A were identified via restriction enzyme digests and verified with DNA sequencing. Human p35 was ordered as a 789 bp geneblock from IDT (Coralville IA) with internal BamH1, BglII and Xba1 sites removed to facilitate cloning. The p35 geneblock was PCR amplified as described above and ligated upstream of the p40 geneblock in P2A-hIL12p40/pOMI2A. Positives clones of hIL12p35-P2A-p40/pOMI2A were identified via restriction enzyme digests and verified with DNA sequencing. pIL-12 P2A is formulated in phosphate buffered saline (PBS) for direct intratumoral injection followed by in vivo EP. GMP-grade pIL-12 P2A is supplied as 2.0 ml vials at a concentration of 0.5 mg/ml and fill volume of 1.5 ml. Unopened vials of pIL-12 P2A are stored in a secure, continuously temperature monitored and alarmed freezer in the pharmacy or other appropriate secure location at −20° C.±5° C.

### Example 2. Clinical Study

Plasmid encoded IL-12 plus electroporation (IT-EP IL-12) in combination with intravenous pembrolizumab therapy with chemotherapy in the treatment of cancer.

TNBC accounts for 10-20% of breast cancer diagnoses. Chemotherapy is the current standard-of-care treatment in the adjuvant, neoadjuvant, and metastatic settings. TNBCs are highly sensitive to chemotherapy, as evidenced by pathologic complete response (pCR) rates in the 30% to 40% range after combination neoadjuvant chemotherapy. However, TNBC has higher rates of relapse and is associated with a disproportionate number of breast cancer deaths, which has been referred to as the triple-negative paradox.

Patients with TNBC could benefit from the addition of immune-based therapy due to the significant role of tumor-infiltrating lymphocytes (TILs) on prognosis. Recent data suggest that TNBC tumors that have a proinflammatory environment are associated with better outcomes, including in the context of chemotherapies like gemcitabine and carboplatin. TNBC patients with at least 50% TILs demonstrate longer disease-free survival when treated with anthracycline and taxane-base adjuvant therapy (Adams S et al. (2014). “Prognostic value of tumor-infiltrating lymphocytes in triple-negative breast cancers from two phase III randomized adjuvant breast cancer trials: ECOG 2197 and ECOG 1199.” Journal of clinical oncology 32(27): 2959-2966; Loi S et al. (2014). “Tumor infiltrating lymphocytes are prognostic in triple negative breast cancer and predictive for trastuzumab benefit in early breast cancer: results from the FinHER trial.” Annals of oncology 25(8): 1544-1550; Loi S et al. (2015). “Pooled individual patient data analysis of stromal tumor infiltrating lymphocytes in primary triple negative breast cancer treated with anthracycline-based chemotherapy.” 38th Annual San Antonio Breast Cancer Symposium; Dec. 8-12, 2015). IT-EP IL-12 can be used to induce a proinflammatory environment in the area of a tumor.

Preliminary data suggests that TNBC patients selected for PD-1 expression have nearly twice the objective anti-tumor responses rate (18.5%) as those who are unselected for PD-L1 expression (<10%; Dua I et al (2017). “Immunotherapy for triple-negative breast cancer: A focus on Immune checkpoint inhibitors.” American Journal of Hematology Oncology 13(4): 20-27). Anti-PD-1/PD-L1 mAbs given as monotherapy have demonstrated limited success in this population and appear to require an immunogenic tumor, characterized by CD8+ TILs and/or PD-L1 expression to be effective.

IT-EP IL-12 causes intratumoral expression of the proinflammatory cytokine IL-12 enabling conversion of poorly-immunogenic/low T-cell infiltrating tumors into highly inflamed immunologically active lesions without significantly increasing circulating IL-12 levels. In patients with advanced cutaneous malignancies (e.g., melanoma), IT-EP IL-12 leads to regression of both treated and untreated lesions while demonstrating a highly-favorable safety profile. In addition, clinical evidence suggests the IT-EP IL-12/anti-PD-1 antibody combination therapy can be effective in providing durable objective tumor responses in patients with immunologically cold tumors (Algazi A. (2017). “Immune monitoring outcomes of patients with stage III/IV melanoma treated with a combination of pembrolizumab and intratumoral plasmid interleukin 12 (pIL-12).” ASCO-SITC Clinical Immuno-Oncology Symposium, poster presentation).

TNBC patients will be treated with IT-EP IL-12 therapy plus checkpoint inhibitor therapy with additional chemotherapy. The checkpoint inhibitor therapy can be, but is not limited to, anti-PD-1/anti-PD-L1 antibody therapy. The anti-PD-1/anti-PD-L1 antibody therapy can be, but is not limited to pembrolizumab therapy. The chemotherapy can be, but is not limited to, taxane therapy (e.g., nab-paclitaxel). Combining immunostimulatory cytokine therapy, immune checkpoint inhibitor therapy, and chemotherapy is anticipated to improve responses for TNBC subjects by increasing the “proinflammatory environment” of tumors and that the combination will have a favorable safety profile.

Eligible subjects will have a pathological confirmed diagnosis of locally advanced or metastatic TNBC. The subjects have estrogen (ER) receptor and progesterone (PR) receptor staining <10% and are human epidermal growth factor receptor 2 (HER2)-negative

Cohort 1: Subjects with previously treated inoperable locally advanced or metastatic TNBC are treated with IT-EP IL-12 in combination with pembrolizumab and efficacy is measured by objective response rate (ORR) assessed the by the Investigator based on Response Evaluation Criteria in Solid Tumors (RECIST) v1.1. Subject in cohort 1 have had at least 1 prior therapy.

Cohort 2: Subjects with inoperable locally advanced or metastatic TNBC are treated with IT-EP IL-12 in combination with pembrolizumab and nab-paclitaxel chemotherapy as a first line treatment. Efficacy is assessed by ORR assessed based on RECIST v1.1. The subject may have received a neoadjuvant and adjuvant treatment in the non-metastatic or operable disease setting.

Duration of response (DOR), ORR, immune ORR (iORR), progression-free survival (PFS), immune PFS (iPFS), disease control rate (DCR), and overall survival (OS) will be followed. Response in PD-L1 positive and negative subjects is monitored. PD-L1 negative patients are estimated to be 35-60% of enrolled population.

Nucleic acid encoding IL-12 (0.5 mg/mL) is injected intratumorally (on days 1, 5 and 8 every 6 weeks, i.e., days 1, 5, and 8 of a six week cycle) at a dose volume of approximately ¼ of the calculated lesion volume with a minimum dose volume per lesion of 0.1 mL for lesions of volume <0.4 cm³. After or concurrently with intratumoral injections, electroporation device (applicator) electrodes are positioned into and/or around the injected tumor. The electrodes are co-localized at the site(s) and depth of nucleic acid injection. In some embodiments, the electroporation device comprises 6 stainless-steel needles (electrodes) positioned in an about 0.5 to about 1 cm diameter circular array. The applicator is connected to a power supply and 6 pulses are at a field strength of about 300 to about 1500 volt/cm and pulse width of about 100 μs to about 5 ms are administered at about 300 msec intervals.

Pembrolizumab is administered at a dose of about 200 mg using a 30-minute (−5 minutes/+10 minutes) intravenous (IV) infusion on Day 1 of every 3 weeks (i.e., day 1 of a 3 week cycle).

In some embodiments, day 1 of the IT-EP IL-12 treatment cycle and day 1 of the pembrolizumab treatment cycle are initiated on the same day.

TABLE 3
Study Treatment Schedule (weeks 1-16).

Cohort 1

Cohort 2

| | IT-EP IL-12 | | Checkpoint * | Nab-paclitaxel * |
|---|---|---|---|---|
| | Days 1, 5 and 8 of | | Day 1 of 3- | Days 1, 8, and 15 |
| Week | 6-week cycle * | | week cycle | of 4-week cycle |
| 1 | Days | 1, 5 | Day 1 | Day 1 |
| 2 | Day | 1 | | Day 1 |
| 3 | | | | Day 1 |
| 4 | | | Day 1 | |
| 5 | | | | Day 1 |
| 6 | | | | Day 1 |
| 7 | Days | 1, 5 | Day 1 | Day 1 |
| 8 | Day | 1 | | |
| 9 | | | | Day 1 |
| 10 | | | Day 1 | Day 1 |
| 11 | | | | Day 1 |
| 12 | | | | |
| 13 | Days | 1, 5 | Day 1 | Day 1 |
| 14 | Day | 1 | | Day 1 |

| | | |
|---|---|---|
| 15 | | Day 1 |
| 16 | Day 1 | |
| . . . | | |

* treatment window ± 2 days

TABLE 4
Investigational Treatment Dosing Regimen.

| | | | Dosage formulation/ | | |
|---|---|---|---|---|---|
| | | | Route of | | Cycle |
| Study Drug | Dose | | administration | Schedule | length |
| IL-12 plasmid | ¼ tumor volume | | 0.5 mg/mL Solution/ | Days 1, 5, 8 | 6 weeks |
| | (0.1 mL minimum) | | Intratumoral | (±2 days) | |
| Pembrolizumab | 200 | mg | Solution/IV infusion | Day 1 | 3 weeks |
| | | | (30 min −5/+10 min) | (±2 days) | |
| Nab-paclitaxel | 100 | mg/m² | Solution/IV infusion | Days 1, 4, 15 | 4 weeks |
| | | | (30 min −5/+10 min) | (±2 days) | |

Cohort 1: IT-EP IL-12 is administered by intratumoral injection at days 1, 5, and 8 every 6 weeks. The subjects may receive IT-EP IL-12 treatment in one or more accessible tumor (lesions). Accessible lesions include cutaneous and subcutaneous lesions. Pembrolizumab is administered intravenously on day 1 every 3 weeks. IT-EP IL-12 and pembrolizumab therapies are initiated on the same day such that the IT-EP IL-12 6 week cycles run concurrently with the pembrolizumab 3 week cycles and patients receive IT-EP IL-12 and pembrolizumab on day 1 every 6 weeks.

Cohort 2: IT-EP IL-12 is administered by intratumoral injection at days 1, 5, and 8 every 6 weeks. The subjects may receive IT-EP IL-12 treatment in one or more accessible tumor (lesions). Accessible lesions include cutaneous and subcutaneous lesions. Pembrolizumab is administered intravenously on day 1 every 3 weeks. IT-EP IL-12 and pembrolizumab therapies are initiated on the same day such that the IT-EP IL-12 6 week cycles run concurrently with the pembrolizumab 3 week cycles and patients receive IT-EP IL-12 and pembrolizumab on day 1 every 6 weeks. In additional, patients are administered nab-paclitaxel chemotherapy according to standard of care in the first line setting, e.g., about 100 mg/m² intravenously on Days 1, 8, and 15 every 4 weeks (28 days) or as indicated on the product label. Patients are administered IT-EP IL-12, pembrolizumab, and nab-paclitaxel when treatment is initiated on day 1.

Subjects are treated with IT-EP IL-12 to the accessible lesions on days 1, 5 and 8 of a six week cycle for up to 17 cycles and with IV pembrolizumab (200 mg) on day 1 of each 3-week cycle for up to 33 cycles of pembrolizumab (approximately 2 years. For Cohort 2, subjects are also treated with nab-paclitaxel) on days 1, 8 and 15 of a 4 week cycle for up to 25 cycles (about 2 years).

IT-EP IL-12 treatment for any lesion may be discontinued if there is complete response for that lesion. If there are no accessible lesions for patients in either Cohort, treatment with pembrolizumab may be continued until either complete response (CR) is confirmed or the subject has received to 33 cycles of pembrolizumab. For patients in Cohort 2, nab-paclitaxel can be continued per Investigator's discretion until either CR is confirmed or subject has received 25 cycles of nab-paclitaxel.

Treatment may continue as long as the subject derives a net benefit from the treatment. Net benefit can be, but is not limited to: complete response, partial response, stable disease, a decrease in the size of one or more lesions, a net decrease in tumor volume, absence of formation of new lesions, or an improvement in one or more cancer associated symptoms.

In additional studies, other antagonist PD-1/PD-L1 therapies or other checkpoint inhibitors can be used in combination with IT-EP IL-12 and administration of nab-paclitaxel.

In further additional studies, other chemotherapies are used in combination with IT-EP IL-12 and immune checkpoint inhibitor therapy.

The various combinations IT-EP IL-12, checkpoint inhibitor, and chemotherapeutic can be used to treat other cancers, including, but not limited to, breast cancer, melanoma, head and neck cancer, squamous cell carcinoma, basal cell carcinoma, and Merkel cell carcinoma.

In further additional studies, the combination of immunostimulatory cytokine therapy (e.g., IT-EP IL-12), checkpoint inhibitor therapy (e.g., pembrolizumab therapy), and chemotherapy (e.g., nab-paclitaxel therapy) is used in a first line therapy to treat breast cancer, TNBC, melanoma, head and neck cancer, squamous cell carcinoma, basal cell carcinoma, or Merkel cell carcinoma. The chemotherapy is selected base of the type of cancer to be treated.

In further additional studies, the combination of immunostimulatory cytokine therapy (e.g., IT-EP IL-12), checkpoint inhibitor therapy (e.g., pembrolizumab therapy), and chemotherapy (e.g., nab-paclitaxel therapy) is used in a second-line therapy to treat breast cancer, TNBC, melanoma, head and neck cancer, squamous cell carcinoma, basal cell carcinoma, or Merkel cell carcinoma. The chemotherapy is selected base of the type of cancer to be treated.

In further additional studies, the combination of immunostimulatory cytokine therapy (e.g., IT-EP IL-12), checkpoint inhibitor therapy (e.g., pembrolizumab therapy), and chemotherapy (e.g., nab-paclitaxel therapy) is used in a second-line therapy to treat breast cancer, TNBC, melanoma, head and neck cancer, squamous cell carcinoma, basal cell carcinoma, or Merkel cell carcinoma. The chemotherapy is selected base of the type of cancer to be treated.

In further additional studies, the combination of immunostimulatory cytokine therapy (e.g., IT-EP IL-12), checkpoint inhibitor therapy (e.g., pembrolizumab therapy), and chemotherapy (e.g., nab-paclitaxel therapy) is used as a neoadjuvant therapy in the treatment of operable breast cancer, TNBC, melanoma, head and neck cancer, squamous cell carcinoma, basal cell carcinoma, or Merkel cell carcinoma. The chemotherapy is selected base of the type of cancer to be treated.

In further additional studies, the combination of immunostimulatory cytokine therapy (e.g., IT-EP IL-12), checkpoint inhibitor therapy (e.g., pembrolizumab therapy), and chemotherapy (e.g., nab-paclitaxel therapy) is used in the treatment of inoperable breast cancer, TNBC, melanoma, head and neck cancer, squamous cell carcinoma, basal cell carcinoma, or Merkel cell carcinoma. The chemotherapy is selected base of the type of cancer to be treated

Response to treatment in this study will be evaluated using the international criteria proposed by the RECIST Committee (Therasse P et al. (2000). “New guidelines to evaluate the response to treatment in solid tumors. European Organization for Research and Treatment of Cancer, National Cancer Institute of the United States, National Cancer Institute of Canada.” Journal of the National Cancer Institute 92(3):205-216), modified in 2009 (Eisenhauer E A et al (2009). “New response evaluation criteria in solid tumours: revised RECIST guideline (version 1.1).” European journal of cancer 45(2):228-247) and clarified for disease-specific adaptation (Schwartz L H et al. (2016). “RECIST 1.1—Standardisation and disease-specific adaptations: Perspectives from the RECIST Working Group.” European journal of cancer 62: 138-145). The primary efficacy endpoint, ORR, will be evaluated using RECIST v1.1.

iRECIST will be used to assess a secondary endpoint according to recently published consensus guidelines for iRECIST (Seymour L et al. (2017). “iRECIST: guidelines for response criteria for use in trials testing immunotherapeutics.” The Lancet. Oncology 18(3): e143-e152). Immunotherapeutics may result in infiltration of immune cells leading to transient increase in the size in malignant lesions, or undetectable lesions becoming detectable. The criteria are identical to those of RECIST v1.1 in many respects but have been adapted to account for instances where an increase in tumor burden, or the appearance of new lesions, does not reflect true tumor progression.

### Example 3

A Phase 2, Multi-Cohort, Open-Label Study of IT-EP IL-12 in combination with intravenous pembrolizumab therapy with chemotherapy in patients with inoperable locally advanced or metastatic TNBC.

This is a Phase 2, Multi-Cohort, Open-Label, Study. Cohort 1 will be a single-arm study of IT-EP IL-12 plus pembrolizumab therapy. Cohort 2 will be a single-arm study of IT-EP IL-12 plus pembrolizumab with nab-paclitaxel (Abraxane®) chemotherapy.

Cohort 1: IT-EP IL-12 will be administered by intralesional injection at day 1, 5, and 8 week 1 then every 6 weeks. Pembrolizumab will be administered intravenously on day 1 week 1 then every 3 weeks. Cohort 1 subjects have previously treated inoperable locally advanced or metastatic TNBC

Cohort 2: IT-EP IL-12 will be administered by intralesional injection at days 1, 5, and 8 week 1 then every 6 weeks. Pembrolizumab will be administered intravenously on day 1 week 1 then every 3 weeks. Nab-paclitaxel (Abraxane®) chemotherapy will be administered standard of care in the first line setting, 100 mg/m² intravenously on days 1, 8, and 15 week 1 and then every 4 weeks (28 days). Cohort 2 is a first line treatment in subjects that have inoperable locally advanced or metastatic TNBC.

Subjects with TNBC and EP accessible cutaneous/subcutaneous disease will be enrolled in this study. Eligible subjects have estrogen (ER) receptor and progesterone (PR) receptor staining <10% and are human epidermal growth factor receptor 2 (HER2)-negative as defined by immunohistochemistry (IHC) 0 to 1+. If IHC is equivocal then fluorescence in situ hybridization (FISH) or in situ hybridization (ISH) negative will be acceptable. Eligible subjects may have received neoadjuvant and adjuvant treatment in the non-metastatic or operable disease setting and must not have progressed within 6 months of last dose of (neo)adjuvant therapy. Subjects are assayed for PD-L1 per Dako 22C3 CPS prior to initiating treatment.

Approximately, 65 subjects will be enrolled; 25 in Cohort 1 and 40 in Cohort 2.

Eligible subjects in cohort 1 will be treated with IT-EP IL-12 to the accessible lesions on days 1, 5 and 8 every 6 weeks and with IV pembrolizumab (200 mg) on day 1 of each 3-week cycle for up to 17 cycles of IT-EP IL-12 and 33 cycles of pembrolizumab from baseline (approximately 2 years) or until subsequent disease progression. For Cohort 2, subjects will also be treated with nab-paclitaxel, an approved chemotherapy, per standard of care.

One or more accessible lesions, each ≥0.3 cm×0.3 cm will be treated. Accessible lesion(s) for IT-EP, include cutaneous or subcutaneous lesions that can be reached from the surface with the EP needle array (up to 1.5 cm depth). Only documented accessible lesions that are in a suitable, safe location for application of EP will be eligible for treatment. The accessible lesions are treated on days 1, 5, and 8 of the 6-week treatment cycle.

For patients in both Cohorts, in the event of absence of accessible lesions as a result of the treatment, treatment with pembrolizumab (cohort 1) and pembrolizumab and nab paclitaxel (cohort 2) will be continued until either complete response (CR) is confirmed or the subject receives up to 33 cycles of pembrolizumab and/or up to 25 cycles of nab-paclitaxel. The decision to continue pembrolizumab or nab-paclitaxel can be assessed independently.

Plasmid encoding IL-12 is administered at a dose of ¼ tumor volume at a concentration of 0.5 mg/mL by IT-EP on days 1, 5, and 8 (±2 days) every 6 weeks. Tumor volume is determined by: ¼ tumor volume=[(longest diameter in cm) ×(perpendicular diameter in cm)2]/8. A minimum of 0.1 mL per lesion is injected for lesions <0.1 cm3 in ¼ volume will be administered. One, more than one, or all accessible target lesions can be treated. The lesions can be, but are not limited to, cutaneous or subcutaneous lesions. Intratumoral injection is distributed throughout the lesions with larger lesions optionally receiving multiple injections. Immediately following injection of the IL-12 plasmid into the tumor, an electroporation applicator tip containing 6 needles in a 0.5-1 cm diameter circular array is positioned into or around the site of injection and inserted to a depth approximating the depth of the injection. The applicator is connected to an electroporation generator and 6 pulses at a field strength of 1500 V/cm and a pulse width of 100 ρs at 300 msec intervals are then delivered to the tumor. Once a tumor has been treated, the next tumor is injected and electroporated. IT-EP IL-12 may be expanded to any newly presenting lesions during the course of treatment. Prior to injection, the subject may be administered 1% lidocaine around the injection site to obtain local anesthesia. The subject may also be given analgesics or anxiolytics prior to or during treatment.

The IL-12 plasmid encoded genes for the human IL-12 p35 and p40 subunits separated by an internal ribosomal entry site (IRES) under the control of a CMV promoter. Optionally, the IL-12 plasmid encodes genes for the human IL-12 p35 and p40 subunits separated by a 2A element.

The IL-12 plasmid is formulated in phosphate buffered saline (PBS) for direct intratumoral injection and delivery to tumor cells via electroporation. Optionally, the IL-12 plasmid is formulated in any solution suitable for injection into a subject.

Pembrolizumab is administered according to the drug product label. In some embodiments, Pembrolizumab is administered IV at the dose of about 200 mg using a 30-minute (−5/+10 minutes) IV infusion on Day 1 (±2 days) of each 3-week cycle. On any give cycle, one or more of the injections may be withheld or administered at a different dose or on a different day as medically necessitated. Altering dosage (administering at a different dose) can comprise discontinuing an infusion, altering the induction rate, pausing and restarting infusion, or administering a difference amount of drug during an infusion. Adverse reactions to pembrolizumab may also be treated according to the manufacture's recommendations (e.g., drug product label).

Nab-paclitaxel is administered as recommended per the approved label: 100 mg/m2 to 260 mg/m2 on days 1, 8, and 15 every four weeks, intravenously over about 30 minutes. In some subjects, nab-paclitaxel is administered at 100 mg/m2.

The study duration for each individual subject in cohort 1 will be up to 33 cycles with pembrolizumab from baseline (approximately 2 years) For cohort 2, duration will be up to 33 cycles with pembrolizumab and/or 25 cycles of nab-paclitaxel from baseline (approximately 2 years).

Alternatively, treatment may be continued if the subject is deriving a net benefit from treatment such as a decrease in the size of one or more lesions, a net decrease in tumor volume or in the setting of new lesions, or an improvement in cancer associated symptoms.

Initial biopsy samples (prior to initiation of treatment) are obtained to determine the frequency of PD-1hiCTLA-4hi cells in the live CD45+CD3+CD8+ gate. In some embodiments, biopsy sample are obtained during the course of treatment to assess PD-1 and immune marker levels in the tumor or tumor microenvironment. Marker levels are determined by chromogenic, multispectral immunohistochemical, or nucleic acid detection methods. Gene expression in the samples can be assessed by NanoString, RNA-seq analyses or epigenetic (such as by Illumina Methylation Assay) analyses. In addition, these analyses can be used to monitor IL-12 expression, changes in infiltrating T cell populations.

Response to treatment can be measured by RECIST (v. 1.1) or (immune-related RECIST (iRECIST). The treatment regimen is administered to provide one or more of: increased progression free survival, increased overall survival, increased disease control rate, increased complete response, increased partial response, increased stable disease, and decreased progressive disease.

Complete Response (CR) is marked by the disappearance of all target lesions determined by two separate observations conducted not less than 4 weeks apart, no appearance of new lesions, and disappearance of all non-target (untreated) lesions and optionally normalization of tumor marker levels.

Partial Response (PR) is marked by at least a 30% decrease in the sum of the longest diameter (LD) of target lesions, taking as reference the baseline sum LD. No appearance of new lesions, and non-target lesions are non-PD.

Stable Disease (SD) is marked by neither sufficient shrinkage to qualify for PR nor sufficient increase to qualify for PD, taking as reference the smallest sum LD since the treatment started and persistence of one or more non-target lesion(s) and/or maintenance of tumor marker level above the normal limits.

Progressive Disease (PD) is marked by at least a 20% increase in the sum of diameters of measured lesions taking as references the smallest sum of diameters recorded on study (including baseline) and an absolute increase of ≥0.5 cm, or the appearance of one or more new lesions.

Progression free survival (PFS) is the time, in months, from the first dosing date until the date of disease progression (i.e., the date of the tumor imaging) or death from any cause. RECIST v1.1 is used to determine the dates of progression.

Disease Control Rate (DCR) is the percentage of subjects with a complete response (CR), partial response (PR), or stable disease (SD) for at least 6 months.

Overall survival (OS) is the time from diagnosis until death from any cause.

The ORR is the best response recorded from the start of the treatment until disease progression/recurrence (taking as reference for progressive disease the smallest measurements recorded since the treatment started) (Table 5).

TABLE 5
Overall Response Definitions using RECIST v1.1

| Target | Non-Target | New | Overall | Best Response for this |
|---|---|---|---|---|
| Lesions | Lesions | Lesions | Response | Category Also Requires |
| CR | CR | No | CR | Normalization of tumor |
| | | | | markers, tumor nodes <1.0 cm |
| CR | Non-CR/Non-PD | No | PR | |
| CR | Not all evaluated | No | PR | |
| PR | Non-PD/No all | No | PR | |
| | evaluated | | | |
| SD | Non-PD | No | SD | Documented at least once ≥4 |
| | | | | weeks from baseline |
| Not all | Non-PD | No | NE | |
| evaluated | | | | |
| PD | Any | Any | PD | |
| Any | PD* | Any | PD | |
| Any | Any | Yes | PD | |

The treatment regimen may be adapted for treatment of other cancers, including, but not limited to, melanoma.

1. A method of treating cancer in a subject, the method comprising:
(a) injecting at least one tumor in the subject with an effective dose of an expression vector encoding an immunostimulatory cytokine and administering electroporation therapy to the tumor;
(b) administering an effective dose of an immune checkpoint inhibitor to the subject; and
(c) administering an effective dose of a chemotherapeutic agent to the subject.

2. The method of claim 1, wherein the immunostimulatory cytokine comprises interleukin-12 (IL-12).

3. The method of claim 2, wherein the expression vector encoding the immunostimulatory cytokine comprises a nucleic acid encoding an IL-12 p35 subunit and an IL-12 p40 subunit.

4. The method of any one of claims 1-3, wherein the immunostimulatory cytokine is injected into the tumor and the electroporation therapy is administered to the tumor on day 1±2 days, day 5±2 days, and day 8±2 days of a 6 week cycle for at least 2 cycles.

5. The method of any one of claims 1-4, wherein administering electroporation therapy to the tumor comprises delivering to the at least one tumor at least one voltage pulse over a duration of about 100 microseconds to about 1 millisecond.

6. The method of claim 5, wherein the at least one voltage pulse has a field strength of about 300 V/cm to about 1500 V/cm.

7. The method of claim 6, wherein the at least one voltage pulse has a field strength of about 350 V/cm and a duration of about 10 msec, or a field strength of about 1500 V/cm and a duration of about 100 psec.

8. The method of any one of claims 5-7, wherein at least one voltage pulse comprises 6-8 pulses.

9. The method of any one of claims 1-8, wherein the immune checkpoint inhibitor comprises a PD-1 or PD-L1 antagonist.

10. The method of claim 9, wherein the PD-1 or PD-L1 antagonist comprises an anti-PD-1 or anti-PD-L1 antibody or antibody fragment.

11. The method of claim 9 or 10, wherein the PD-1 or PD-L1 antagonist is administered systemically.

12. The method of claim 11, wherein the PD-1 or PD-L1 antagonist is selected from the group consisting of: nivolumab, pembrolizumab, pidilizumab, durvalumab, atezolizumab, avelumab, cemiplimab, sintilimab, toripalimab, and camrelizumab.

13. The method of any one of claims 1-12, wherein the immune checkpoint inhibitor is administered to the subject on day 1±2 days of a 3 week cycle for at least 2 cycles.

14. The method of any one of claims 1-13, wherein the chemotherapeutic agent is administered systemically.

15. The method claim 14, wherein the chemotherapeutic agent comprises paclitaxel or nab-paclitaxel.

16. The method claim 15, wherein the paclitaxel or nab-paclitaxel is administered to the subject on day 1±2 days, day 8±2 days, and day 15±2 days of a 4 week cycle for at least 2 cycles.

17. The method of any one of claims 1-16, wherein claim 1, wherein injecting the at least one tumor with the expression vector encoding the immunostimulatory cytokine; administering the immune checkpoint inhibitor; and administering chemotherapeutic agent are initiated on the same day.

18. The method of any one of claims 1-17, wherein the tumor is a cutaneous subcutaneous tumor.

19. The method of any one of claims 1-18, wherein the cancer is operable.

20. The method of any one of claims 1-18, wherein the cancer is inoperable.

20. The method of claim 18 or 19, wherein the cancer is locally advanced, recurrent, or metastatic.

21. The method of any one of claims 1-20, wherein the subject has not received one or more prior cancer therapies.

22. The method of any one of claims 1-20, wherein the subject has received one or more prior cancer therapies.

23. The method of claim 20, where the subject that has received prior neoadjuvant or adjuvant treatment in a non-metastatic or potentially operable disease setting.

24. The method of 1-19, wherein the therapy is administered as a neoadjuvant.

25. The method of any one of claims 1-24, wherein the cancer is selected from the group consisting of: melanoma, Merkel cell carcinoma, basal cell carcinoma, squamous cell carcinoma, breast cancer, triple negative breast cancer, and head and neck cancer.

26. The method of claim 17, wherein the cancer is triple negative breast cancer.

27. The method of claim 17, wherein the triple negative breast cancer is inoperable locally advanced or metastatic TNBC.

28. The method of claim 1, wherein the method results in a decrease in the size of one or more tumors, a decrease in formation of new tumors, or an improvement in one or more cancer-associated symptom, an increase disease-free, an increase in survival, an increase in progression free survival, or an increased disease control rate.

29. The method of any one of claims 1-17, wherein the immunostimulatory cytokine comprises IL-12, the immune checkpoint inhibitor comprises an anti-PD-1 or anti-PD-L1 antibody, the chemotherapeutic agent comprises nab-paclitaxel, and the subject is diagnosed with inoperable locally advanced or metastatic triple negative breast cancer.

30. The method of claim 29, wherein the anti-PD-1 or anti-PD-L1 antibody comprises pembrolizumab.

31. The method of claim 30, wherein the IL-12 is administered by IT-EP on days 1±2 days, day 5±2 days, and day 8±2 days of a 6 week cycle for at least 2 cycles, the pembrolizumab is administered systemically on day 1±2 days of a 3 week cycle for at least 2 cycles, and the nab-paclitaxel 1±2 days, day 8±2 days, and day 15±2 days of a 4 week cycle for at least 2 cycles.
